SEQUENCE LISTING (16 sequences)

SEQ ID NO: 1 - protein, 856 aa, Human immunodeficiency virus
  MISC_FEATURE: gp160 of HIV-1 isolate HXB2
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 615 aa, Artificial Sequence
  HIV Env consensus clade C
NLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNNNNMKEEMKNCSFNTTTEIRDKKQKEYALFYRLDIVPLNENSSEYRLINCNTSTITQACPKVSFDPIPIHYCAPAGYAILKCNNKTFNGTGPCNNVSTVQCTHGIKPVVSTQLLLNGSLAEEEIIIRSENLTDNAKTIIVHLNESVEINCTRPNNNTRKSIRIGPGQTFYATGDIIGDIRQAHCNISEAKWNKTLQRVKKKLKEHFPNKTIKFAPSSGGDLEITTHSFNCRGEFFYCNTSKLFNSTYNNTTSNSTITLPCRIKQIINMWQEVGRAMYAPPIAGNITCKSNITGLLLTRDGGNNNNNTETFRPGGGDMRDNWRSELYKYKVVEIKPLGIAPTKAKRRVVEREKRRAVGIGAVFLGFLGAAGSTMGAASITLTVQARQLLSGIVQQQSNLLRAIEAQQHMLQLTVWGIKQLQARVLAIERYLKDQQLLGIWGCSGKLICTTAVPWNSSWSNKSQEDIWDNMTWMQWDREISNYTDTIYRLLEESQNQQEKNEKDLLALD

SEQ ID NO: 3 - protein, 677 aa, Artificial Sequence
  ConC_SOSIP sequence
NLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNNNNMKEEMKNCSFNTTTEIRDKKQKEYALFYRLDIVPLNENSSEYRLINCNTSTITQACPKVSFDPIPIHYCAPAGYAILKCNNKTFNGTGPCNNVSTVQCTHGIKPVVSTQLLLNGSLAEEEIIIRSENLTDNAKTIIVHLNESVEINCTRPNNNTRKSIRIGPGQTFYATGDIIGDIRQAHCNISEAKWNKTLQRVKKKLKEHFPNKTIKFAPSSGGDLEITTHSFNCRGEFFYCNTSKLFNSTYNNTTSNSTITLPCRIKQIINMWQEVGRAMYAPPIAGNITCKSNITGLLLTRDGGNNNNNTETFRPGGGDMRDNWRSELYKYKVVEIKPLGIAPTKCKRRVVERRRRRRAVGIGAVFLGFLGAAGSTMGAASITLTVQARQLLSGIVQQQSNLLRAPEAQQHMLQLTVWGIKQLQARVLAIERYLKDQQLLGIWGCSGKLICCTAVPWNSSWSNKSQEDIWDNMTWMQWDREISNYTDTIYRLLEESQNQQEKNEKDLLALDAAALPETGGGSDYKDDDDKPGGGGSGGGGSGGGGSGGGGSGGGGSGGGGSGGGGSHHHHHH

SEQ ID NO: 4 - protein, 630 aa, Artificial Sequence
  HIV Env consensus clade B
AEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNNNTTNNNSSSEKMEKGEIKNCSFNITTSIRDKVQKEYALFYKLDVVPIDNNNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIVFNQSSGGDPEIVMHSFNCGGEFFYCNTTQLFNSTWNSNGTWNNTTGNDTITLPCRIKQIINMWQEVGKAMYAPPIRGQIRCSSNITGLLLTRDGGNNNNNTTETFRPGGGDMRDNWRSELYKYKVVKIEPLGVAPTKCKRRVVQRRRRRRAVGIGAMFLGFLGAAGSTMGAASITLTVQARQLLSGIVQQQNNLLRAPEAQQHLLQLTVWGIKQLQARVLAVERYLKDQQLLGIWGCSGKLICCTAVPWNTSWSNKSLDEIWDNMTWMQWEREIDNYTGLIYTLIEESQNQQEKNEQELLELD

SEQ ID NO: 5 - protein, 691 aa, Artificial Sequence
  ConB_SOSIP sequence
AEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLNNNTTNNNSSSEKMEKGEIKNCSFNITTSIRDKVQKEYALFYKLDVVPIDNNNTSYRLISCNTSVITQACPKVSFEPIPIHYCAPAGFAILKCNDKKFNGTGPCTNVSTVQCTHGIRPVVSTQLLLNGSLAEEEVVIRSENFTDNAKTIIVQLNESVEINCTRPNNNTRKSIHIGPGRAFYATGDIIGDIRQAHCNISRTKWNNTLKQIVKKLREQFGNKTIVFNQSSGGDPEIVMHSFNCGGEFFYCNTTQLFNSTWNSNGTWNNTTGNDTITLPCRIKQIINMWQEVGKAMYAPPIRGQIRCSSNITGLLLTRDGGNNNNNTTETFRPGGGDMRDNWRSELYKYKVVKIEPLGVAPTKCKRRVVQRRRRRRAVGIGAMFLGFLGAAGSTMGAASITLTVQARQLLSGIVQQQNNLLRAPEAQQHLLQLTVWGIKQLQARVLAVERYLKDQQLLGIWGCSGKLICCTAVPWNTSWSNKSLDEIWDNMTWMQWEREIDNYTGLIYTLIEESQNQQEKNEQELLELDAAALPETGGGSDYKDDDDKPGGGGSGGGGSGGGGSGGGGSGGGGSGGGGSGGGGSHHHHHH

SEQ ID NO: 6 - protein, 6 aa, Artificial Sequence
  furin cleavage site mutant sequence
RRRRRR

SEQ ID NO: 7 - protein, 31 aa, Artificial Sequence
  signal sequence
MRVRGILRNWQQWWIWGILGFWMLMICNVVG

SEQ ID NO: 8 - protein, 29 aa, Artificial Sequence
  signal sequence
MRVKGIRKNYQHLWRWGTMLLGMLMICSA

SEQ ID NO: 9 - protein, 8 aa, Artificial Sequence
  example of 8 amino acid sequence that can replace HR1 loop
NPDWLPDM

SEQ ID NO: 10 - protein, 8 aa, Artificial Sequence
  example of 8 amino acid sequence that can replace HR1 loop
GSGSGSGS

SEQ ID NO: 11 - protein, 8 aa, Artificial Sequence
  example of 8 amino acid sequence that can replace HR1 loop
DDVHPDWD

SEQ ID NO: 12 - protein, 8 aa, Artificial Sequence
  example of 8 amino acid sequence that can replace HR1 loop
RDTFALMM

SEQ ID NO: 13 - protein, 8 aa, Artificial Sequence
  example of 8 amino acid sequence that can replace HR1 loop
DEEKVMDF

SEQ ID NO: 14 - protein, 8 aa, Artificial Sequence
  example of 8 amino acid sequence that can replace HR1 loop
DEDPHWDP

SEQ ID NO: 15 - protein, 61 aa, Artificial Sequence
  sortase A-Flag-His tag
AAALPETGGGSDYKDDDDKPGGGGSGGGGSGGGGSGGGGSGGGGSGGGGSGGGGSHHHHHH

SEQ ID NO: 16 - protein, 844 aa, Artificial Sequence
  full length ConC_SOSIP
MRVRGILRNWQQWWIWGILGFWMLMICNVVGNLWVTVYYGVPVWKEAKTTLFCASDAKAYEKEVHNVWATHACVPTDPNPQEMVLENVTENFNMWKNDMVDQMHEDIISLWDQSLKPCVKLTPLCVTLNCTNVNVTNTNNNNMKEEMKNCSFNTTTEIRDKKQKEYALFYRLDIVPLNENSSEYRLINCNTSTITQACPKVSFDPIPIHYCAPAGYAILKCNNKTFNGTGPCNNVSTVQCTHGIKPVVSTQLLLNGSLAEEEIIIRSENLTDNAKTIIVHLNESVEINCTRPNNNTRKSIRIGPGQTFYATGDIIGDIRQAHCNISEAKWNKTLQRVKKKLKEHFPNKTIKFAPSSGGDLEITTHSFNCRGEFFYCNTSKLFNSTYNNTTSNSTITLPCRIKQIINMWQEVGRAMYAPPIAGNITCKSNITGLLLTRDGGNNNNNTETFRPGGGDMRDNWRSELYKYKVVEIKPLGIAPTKCKRRVVEREKRAVGIGAVFLGFLGAAGSTMGAASITLTVQARQLLSGIVQQQSNLLRAPEAQQHMLQLTVWGIKQLQARVLAIERYLKDQQLLGIWGCSGKLICCTAVPWNSSWSNKSQEDIWDNMTWMQWDREISNYTDTIYRLLEESQNQQEKNEKDLLALDSWNNLWNWFDITNWLWYIKIFIMIVGGLIGLRIIFAVLSIVNRVRQGYSPLSFQTLTPNPRGPDRLGRIEEEGGEQDRDRSIRLVSGFLALAWDDLRSLCLFSYHRLRDFILIAARAVELLGRSSLRGLQRGWEALKYLGSLVQYWGLELKKSAISLLDTIAIAVAEGTDRIIELIQRICRAIRNIPRRIRQGFEAALL

(19) United States
(12) Patent Application Publication
(10) Pub. No.: US 2022/0265813 A1
(43) Pub. Date: Aug. 25, 2022

(54) Trimer Stabilizing HIV Envelope Protein Mutation

(71) Applicant: Janssen Vaccines & Prevention B.V., Leiden (NL)

(72) Inventors: Johannes Petrus Maria Langedijk, Amsterdam (NL); Jaroslaw Juraszek, Den Haag (NL); Lucy Rutten, Rijnsburg (NL)

(21) Appl. No.: 17/651,973

(22) Filed: Feb. 22, 2022

(30) Foreign Application Priority Data
Feb. 23, 2021 (EP) 21158800.9

(51) Int. Cl. A61K 39/21; A61P 31/18; A61P 37/04; C12N 7/00

(52) U.S. Cl. CPC A61K 39/21; A61P 31/18; A61P 37/04; C12N 7/00; A61K 2039/53

(57) ABSTRACT

Human immunodeficiency virus (HIV) envelope proteins having specified mutations that stabilize the trimeric form of the envelope protein are provided. The HIV envelope proteins described herein have an improved percentage of trimer formation and/or an improved trimer yield. Also provided are particles displaying the HIV envelope proteins, nucleic acid molecules and vectors encoding the HIV envelope proteins, as well as compositions containing the HIV envelope proteins, particles, nucleic acid, or vectors.

### BACKGROUND OF THE INVENTION

Human Immunodeficiency Virus (HIV) affects millions of people worldwide, and the prevention of HIV through an efficacious vaccine remains a very high priority, even in an era of widespread antiretroviral treatment. Antigenic diversity between different strains and clades of the HIV virus renders it difficult to develop vaccines with broad efficacy. HIV-1 is the most common and pathogenic strain of the virus, with more than 90% of HIV/AIDS cases deriving from infection with HIV-1 group M. The M group is subdivided further into clades or subtypes, of which clade C is the largest. An efficacious vaccine ideally would be capable of eliciting both potent cellular responses and broadly neutralizing antibodies capable of neutralizing HIV-1 strains from different clades.

The envelope protein spike (Env) on the HIV surface is composed of a trimer of heterodimers of glycoproteins gp120 and gp41 (FIG. 1A). The precursor protein gp160 is cleaved by furin into gp120, which is the head of the spike and contains the CD4 receptor binding site as well as the large hypervariable loops (V1 to V5), and gp41 that is the membrane-anchored stem of the envelope protein spike. Like other class I fusogenic proteins, gp41 contains an N-terminal fusion peptide (FP), a C-terminal transmembrane (TM) domain, and a cytoplasmic domain. Membrane fusion between HIV and target cell membranes requires a series of conformational changes in the envelope protein. HIV vaccines can be developed based upon the envelope protein.

However, various factors make the development of an HIV vaccine based upon the envelope protein challenging, including the high genetic variability of HIV-1, the dense carbohydrate coat of the envelope protein, and the relatively dynamic and labile nature of the envelope protein spike structure. The wild-type envelope protein is unstable due to its function. Therefore, stabilizing modifications are sometimes introduced into the envelope structure for generating vaccine candidates. The envelope protein is a target for neutralizing antibodies and is highly glycosylated, which reduces the immunogenicity by shielding protein epitopes. All known broadly neutralizing antibodies (bNAbs) do accommodate these glycans.

For vaccine development, it is preferred to use envelope proteins that can induce bNAbs. However, most bNAbs only recognize the native envelope protein conformation before it undergoes any conformation changes. Therefore, developing a stable envelope protein in its native-like compact and closed conformation, while minimizing the presentation of non-native and thus non-neutralizing epitopes, could improve the efficiency of generating such bNAbs. Previous efforts to produce an HIV vaccine have focused on developing vaccines that contain the pre-fusion ectodomain of the trimeric HIV envelope protein, gp140. Gp140 does not have the transmembrane (TM) and cytoplasmic domains, but unlike gp120, it can form trimer structures. Moreover, these previous efforts have mainly focused on clade A. However, the breadth of the neutralizing antibody response that has been induced is still limited. Therefore, it would also be beneficial if stabilized native envelope trimers against multiple HIV clades were available.

For more than two decades, attempts have been made to develop a stable envelope protein in its pre-fusion trimer conformation with only limited success in producing soluble, stable trimers of the envelope protein capable of inducing a broadly neutralizing antibody response. For example, the so-called SOSIP mutations (501C, 605C and 559P) have been introduced into the envelope protein sequence to improve the formation of a soluble gp140 trimer fraction (Sanders et al., (2002), J. Virol. 76(17): 8875-89). The so-called SOSIP mutations include cysteine residues at positions 501 and 605, and a proline residue at position 559 according to the numbering in gp160 of HIV-1 isolate HXB2, which is the conventional numbering scheme used in the field. The introduction of the two cysteine residues at positions 501 and 605, which are close to one another in the three-dimensional protein structure results in a disulfide bridge. SOSIP mutant envelope proteins, such as BG505_SOSIP and B41_SOSIP (envelope proteins from HIV strains BG505 and B41 (i.e. 9032-08.A1.4685) strains with SOSIP mutations), have been used in vaccine studies and shown to induce tier 2 autologous neutralizing Abs (Sanders et al., Science (2015), 349(6224): 139-140).

However, even though the so-called SOSIP mutations are capable of stabilizing the trimer form of the envelope protein, the trimer fraction of such SOSIP mutants is usually below 10%, with large amounts of monomer and aggregates still produced. Even the SOSIP mutant BG505_SOSIP, which is a promising SOSIP mutant envelope in terms of its ability to stabilize the trimer form typically yields up to only 25% of the trimer form (Julien et al., Proc. Nat. Acad. Sci. (2015), 112(38), 11947-52). Moreover, in this trimer fraction the trimers are not completely stable as they breathe at the apex. Thus, in addition to the SOSIP mutations, several additional substitutions, such as E64K, A316W, and 201C-433C, have been designed to stabilize the apex and prevent it from breathing (de Taeye et al., Cell (2015), 163(7), 1702-15; Kwon et al., (2015) Nat. Struct. Mol. Biol. 22(7) 522-31). In addition, further mutations and strategies have been reported to improve trimerization yields and optimize folding and stability of prefusion-closed HIV envelope trimers (WO 2018/050747; WO 2019/016062; Rutten et al, (2018) Cell Reports 23: 584-595; Rawi et al, (2020) Cell Reports 33, 108432).

Accordingly, there is a need for stabilized trimers of HIV envelope proteins that have improved percentage of trimer formation, improved trimer yield, and/or improved trimer stability. Preferably, such stabilized trimers of HIV envelope proteins would also display good binding with broadly neutralizing antibodies (bNAbs), and relatively limited binding to non-broadly neutralizing Abs (non-bNAbs). It is an object of the invention to provide HIV Env proteins that have improved trimer percentages, and preferably also improved trimer yields.

### BRIEF SUMMARY OF THE INVENTION

The invention relates to recombinant HIV envelope proteins that have improved percentage of trimer formation and/or improved trimer yields as compared to certain previously described HIV envelope trimers. The resulting stable and well-folded HIV Env trimers are useful for immunization purposes, e.g. to improve chances of inducing broadly neutralizing antibodies and reducing induction of non-neutralizing and weakly neutralizing antibodies upon administration of the recombinant HIV Env trimers. The invention also relates to isolated nucleic acid molecules and vectors encoding the recombinant HIV envelope proteins, cells comprising the same, and compositions of the recombinant HIV envelope protein, nucleic acid molecule, vector, and/or cells.

In one general aspect, the invention relates to a recombinant human immunodeficiency virus (HIV) envelope (Env) protein comprising one of the amino acids tryptophan (Trp), phenylalanine (Phe), methionine (Met), or leucine (Leu), preferably Trp or Phe at position 650, wherein the numbering of the positions is according to the numbering in gp160 of HIV-1 isolate HXB2. In certain embodiments, such HIV Env proteins further comprise one or more mutations that increase trimer yield and/or stabilize trimers, as indicated herein. Such Env proteins have not been described before, and the Trp, Phe, Met, or Leu amino acid at position 650 leads to increased trimer yields. This has been shown herein as compared to Env proteins having the original amino acid most abundantly found at that position (being glutamine, Gln, Q), both for a Glade B and for a clade C derived Env protein.

In certain preferred embodiments, the HIV Env protein of the invention comprises Trp at position 650.

In certain preferred embodiments, the HIV Env protein of the invention comprises Phe at position 650.

In certain embodiments, a recombinant HIV envelope (Env) protein of the invention further comprises one or more of the following amino acid residues at the indicated positions:
(i) Phe, Leu, Met, or Trp, preferably Phe, at position 651;
(ii) Phe, Ile, Met, or Trp, preferably Ile, at position 655;
(iii) Asn or Gln, preferably Asn, at position 535;
(iv) Val, Ile or Ala at position 589;
(v) Phe or Trp, preferably Phe, at position 573;
(vi) Ile at position 204;
(vii) Phe, Met, or Ile, preferably Phe, at position 647;
(viii) Val, Ile, Phe, Met, Ala, or Leu, preferably Val or Ile, more preferably Val, at position 658;
(ix) Gln, Glu, Ile, Met, Val, Trp, or Phe, preferably Gln or Glu, at position 588;
(x) Lys at position 64 or Arg at position 66 or Lys at position 64 and Arg at position 66;
(xi) Trp at position 316;
(xii) Cys at both positions 201 and 433;
(xiii) Pro at position 556 or 558 or at both positions 556 and 558;
(xiv) replacement of the loop at amino acid positions 548-568 (HR1-loop) by a loop having 7-10 amino acids, preferably a loop of 8 amino acids, for example having a sequence chosen from any one of (SEQ ID NOs: 9-14);
(xv) Gly at position 568, or Gly at position 569, or Gly at position 636, or Gly at both positions 568 and 636, or Gly at both positions 569 and 636;
(xvi) Tyr at position 302, or Arg at position 519, or Arg at position 520, or Tyr at position 302 and Arg at position 519, or Tyr at position 302 and Arg at position 520, or Tyr at position 302 and Arg at both positions 519 and 520;
(xvii) a mutation in a furin cleavage sequence of the HIV Env protein, preferably a replacement at positions 508-511 by RRRRRR (SEQ ID NO: 6);
(xviii) Cys at positions 501 and 605 or Pro at position 559, preferably Cys at positions 501 and 605 and Pro at position 559;
(xix) His at position 108; and/or
(xx) His at position 538,
wherein the numbering of the positions is according to the numbering in gp160 of HIV-1 isolate HXB2. In certain embodiments, an HIV Env protein of the invention comprises the indicated amino acid residues at at least two of the indicated positions selected from the group consisting of (i) to (viii) above.

In certain embodiments, a recombinant HIV Env protein of the invention comprises His at position 108, or His at position 538, or His at position 108 and His at position 538.

In certain embodiments, a recombinant HIV Env protein of the invention comprises Trp, Phe, Met, or Leu, preferably Trp or Phe, at position 650 and further comprises (a) Cys at positions 501 and 605, or (b) Pro at position 559, or preferably (c) Cys at positions 501 and 605 and Pro at position 559 (a so-called ‘SOSIP’ variant HIV Env protein), wherein the numbering of the positions is according to the numbering in gp160 of HIV-1 isolate HXB2. In certain embodiments, this is combined with His at position 108 and/or His at position 538. In certain embodiments, this is combined with one or more of the amino acids at positions described in (i)-(viii) above.

In certain embodiments, a recombinant HIV Env protein according to the invention is from a clade C HIV. In certain embodiments, a recombinant HIV Env protein according to the invention is from a clade B HIV. In certain embodiments, a recombinant HIV Env protein according to the invention is from a clade A HIV. In certain embodiments, a recombinant HIV Env protein according to the invention is from a clade D, E, F, G, H, I, J, K, or L HIV. In certain embodiments, a recombinant HIV Env protein according to the invention is from a circulating recombinant form (CRF) of HIV from two or more of clades A, B, C, D, E, F, G, H, I, J, K, or L.

In certain embodiments, a recombinant HIV Env protein of the invention further comprises a mutation in the furin cleavage sequence of the HIV Env protein, such as a replacement at positions 508-511 by RRRRRR (SEQ ID NO: 6).

In one embodiment, the recombinant HIV Env protein is a gp140 protein.

In another embodiment, the recombinant HIV Env protein is a gp160 protein.

In certain embodiments, the recombinant HIV Env protein is truncated in the cytoplasmic region. In certain embodiments thereof, the truncation is after 7 amino acids of the cytoplasmic region.

Also disclosed are a recombinant HIV Env protein comprising an amino acid sequence that is at least 80%, 85%, 90%, 91%, 92%, 93%, 94%, 95%, 96%, 97%, 98%, 99% or 100% identical to any one of SEQ ID NOs: 2, 3, 4, 5, 16 and wherein the amino acid at position 650 is Trp, Phe, Met, or Leu, preferably Trp or Phe. In this aspect, position 650 is not taken into account when determining the % identity, and wherein numbering is according to numbering in gp160 of HIV-1 isolate HXB2. Also in this aspect, one or more of the amino acids at the indicated positions that are not taken into account for determining the % identity, are preferably chosen from the amino acids indicated as being preferred herein in (i)-(xx) mentioned above.

In another general aspect, the invention relates to a trimeric complex comprising a noncovalent oligomer of three of any of the recombinant HIV Env proteins described herein.

In another general aspect, the invention relates to a particle, e.g. a liposome or a nanoparticle, e.g. a self-assembling nanoparticle, displaying on its surface a recombinant HIV Env protein of the invention, or a trimeric complex of the invention.

In another general aspect, the invention relates to an isolated nucleic acid molecule encoding a recombinant HIV Env protein of the invention.

In another general aspect, the invention relates to vectors comprising the isolated nucleic acid molecule operably linked to a promoter. In one embodiment, the vector is a viral vector. In another embodiment, the vector is an expression vector. In one preferred embodiment, the viral vector is an adenovirus vector.

Another general aspect relates to a host cell comprising the isolated nucleic acid molecule or vector encoding the recombinant HIV Env protein of the invention. Such host cells can be used for recombinant protein production, recombinant protein expression, or the production of viral particles, such as recombinant adenovirus.

Another general aspect relates to methods of producing a recombinant HIV Env protein, comprising growing a host cell comprising an isolated nucleic acid molecule or vector encoding the recombinant HIV Env protein of the invention under conditions suitable for production of the recombinant HIV Env protein.

Yet another general aspect relates to a composition comprising a recombinant HIV Env protein, trimeric complex, isolated nucleic acid molecule, or vector as described herein, and a pharmaceutically acceptable carrier.

In another general aspect, the invention relates to a method of improving the trimer formation of an HIV Env protein, the method comprising substituting an amino acid residue at position 650 in a parent HIV Env protein by Trp, Phe, Met, or Leu, preferably Trp or Phe, wherein the numbering of the positions is according to the numbering in gp160 of HIV-1 isolate HXB2.

### BRIEF DESCRIPTION OF THE FIGURES

The foregoing summary, as well as the following detailed description of the invention, will be better understood when read in conjunction with the appended drawings. It should be understood that the invention is not limited to the precise embodiments shown in the drawings.

FIGS. 1A and 1B show that mutation Q650W increases trimer yield of ConC_SOSIP. FIG. 1A) Analytical SEC with Expi293F cell culture supernatants after transfection with plasmids coding for HIV Env ConC-SOSIP and its Q650W variant. FIG. 1B) AlphaLISA binding of the cell culture supernatants with HIV Env-specific bNAbs and non-bNAbs to ConC_SOSIP and its Q650W variant. All measurements were performed in triplicate.

FIGS. 2A and 2B show that mutation Q650W increases trimer yield of ConB_SOSIP. FIG. 2A) Analytical SEC with Expi293F cell culture supernatants after transfection with plasmids coding for HIV Env ConB-SOSIP and its Q650W variant. FIG. 2B) AlphaLISA binding of the cell culture supernatants with HIV Env-specific bNAbs and non-bNAbs to ConB_SOSIP and its Q650W variant. All measurements were performed in triplicate.

FIG. 3 shows that mutations Q650F, Q650M, and Q650L increase trimer yield of ConC_SOSIP, whereas mutation Q650I decreases trimer formation in ConC_SOSIP, in analytical SEC with Expi293F cell culture supernatants.

FIGS. 4A and 4B show that mutation T538H increases trimer yield of ConC_SOSIP. FIG. 4A) Analytical SEC with Expi293F cell culture supernatants after transfection with plasmids coding for HIV Env ConC-SOSIP and its T538H variant. FIG. 4B) AlphaLISA binding of the cell culture supernatants with HIV Env-specific bNAbs and non-bNAbs to ConC_SOSIP and its T538H variant. All measurements were performed in triplicate.

FIGS. 5A and 5B show that mutation T538H increases trimer yield of ConB_SOSIP. FIG. 5A) Analytical SEC with Expi293F cell culture supernatants after transfection with plasmids coding for HIV Env ConB-SOSIP and its T538H variant. FIG. 5B) AlphaLISA binding of the cell culture supernatants with HIV Env-specific bNAbs and non-bNAbs to ConB_SOSIP and its T538H variant. All measurements were performed in triplicate.

FIGS. 6A and 6B show that mutation I108H increases trimer yield of ConC_SOSIP. FIG. 6A) Analytical SEC with Expi293F cell culture supernatants after transfection with plasmids coding for HIV Env ConC-SOSIP and its I108H variant. FIG. 6B) AlphaLISA binding of the cell culture supernatants with HIV Env-specific bNAbs and non-bNAbs to ConC_SOSIP and its I108H variant. All measurements were performed in triplicate.

FIGS. 7A and 7B show that mutation I108H increases trimer yield of ConB_SOSIP. FIG. 7A) Analytical SEC with Expi293F cell culture supernatants after transfection with plasmids coding for HIV Env ConB-SOSIP and its I108H variant. FIG. 7B) AlphaLISA binding of the cell culture supernatants with HIV Env-specific bNAbs and non-bNAbs to ConB_SOSIP and its I108H variant. All measurements were performed in triplicate.

FIGS. 8A and 8B show that mutations I108H, T538H, and Q650W increase trimer yield as compared to ConcB_SOSIP comprising only the I108H mutation. FIG. 8A) Analytical SEC with Expi293F cell culture supernatants after transfection with plasmids coding for HIV Env ConB-SOSIP comprising the I108H, T538H, and Q650W mutations and HIV Env ConB-SOSIP comprising only the I108H mutation. FIG. 8B) AlphaLISA binding of the cell culture supernatants with HIV Env-specific bNAbs and non-bNAbs to ConB_SOSIP_I108H_T538H_Q650W and ConB_SOSIP_I108H variant. All measurements were performed in triplicate.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. As used herein, the use of a numerical range expressly includes all possible subranges, all individual numerical values within that range, including integers within such ranges and fractions of the values unless the context clearly indicates otherwise.

Amino acids are referenced throughout the disclosure. There are twenty naturally occurring amino acids, as well as many non-naturally occurring amino acids. Each known amino acid, including both natural and non-natural amino acids, has a full name, an abbreviated one letter code, and an abbreviated three letter code, all of which are well known to those of ordinary skill in the art. For example, the three and one letter abbreviated codes used for the twenty naturally occurring amino acids are as follows: alanine (Ala; A), arginine (Arg; R), aspartic acid (Asp; D), asparagine (Asn; N), cysteine (Cys; C), glycine (Gly; G), glutamic acid (Glu; E), glutamine (Gln; Q), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y) and valine (Val; V). Amino acids can be referred to by their full name, one letter abbreviated code, or three letter abbreviated code.

Unless the context clearly dictates otherwise, the numbering of positions in the amino acid sequence of an HIV envelope protein as used herein is according to the numbering in gp160 of HIV-1 isolate HXB2 as for instance set forth in Korber et al. (Human Retroviruses and AIDS 1998: A Compilation and Analysis of Nucleic Acid and Amino Acid Sequences. Korber et al., Eds. Theoretical Biology and Biophysics Group, Los Alamos National Laboratory, Los Alamos, N. Mex.), which is incorporated by reference herein in its entirety. Numbering according to HXB2 is conventional in the field of HIV Env proteins. The gp160 of HIV-1 isolate HXB2 has the amino acid sequence shown in SEQ ID NO: 1. Alignment of an HIV Env sequence of interest with this sequence can be used to find the corresponding amino acid numbering in the sequence of interest.

The term “percent (%) sequence identity” or “% identity” describes the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 95%, 97% or 98% identity) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g using the NCBI BLAST algorithm (Altschul S F, et al (1997), Nucleic Acids Res. 25:3389-3402).

A ‘corresponding position’ in a HIV Env protein refers to position of the amino acid residue when at least two HIV Env sequences are aligned. Unless otherwise indicated, amino acid position numbering for these purposes is according to numbering in gp160 of HIV-1 isolate HXB2, as customary in the field.

The ‘mutation according to the invention’ as used herein is a substitution of the amino acid at position 650 in a parent HIV Env protein by a tryptophan (Trp), phenylalanine (Phe), methionine (Met), or leucine (Leu) residue. Of these, substitution by Trp or Phe are preferred. An additional ‘stabilizing mutation’ as used herein is a mutation as described herein in any of entries (i)-(xvi) of Table 1, which increases the percentage of trimer and/or the trimer yield (which can for instance be measured according to AlphaLISA or size exclusion chromatography (SEC) assays, e.g. analytical SEC assays described herein, or SEC-MALS as described e.g. in WO 2019/016062) of an HIV Env protein as compared to a parent molecule when the mutation is introduced by substitution of the corresponding amino acid in said parent molecule (see e.g. WO 2019/016062). Other novel stabilizing mutations that can optionally be combined with the mutation according to the invention is a substitution of the amino acid at position 108 in a parent HIV Env protein by a histidine (His) residue, or a substitution of the amino acid at position 538 in a parent HIV Env protein by a histidine (His) residue, or substitutions of the amino acids at both positions 108 and 538 by His residues. The amino acids resulting from such stabilizing mutations typically are rarely, if at all, found in Env proteins of wild-type HIV isolates.

In another aspect, the invention provides for a HIV Env protein comprising histidine (His) at position 108, wherein the numbering of the positions is according to the numbering in gp160 of HIV-1 isolate HXB2. Such Env proteins have not been described before, and the His amino acid at position 108 leads to increased trimer yields. This has been shown herein as compared to Env proteins having the original amino acid most abundantly found at that position (being isoleucine, Ile), both for a clade B and for a clade C derived Env protein. The HIV Env protein comprising histidine (His) at position 108 can be optionally combined with the 650 and/or 538 modifications or any of the other amino acid modifications as described herein. In certain embodiments, a recombinant HIV Env protein of the invention comprises His at position 108 and further comprises (a) Cys at positions 501 and 605, or (b) Pro at position 559, or preferably (c) Cys at positions 501 and 605 and Pro at position 559, wherein the numbering of the positions is according to the numbering in gp160 of HIV-1 isolate HXB2.

The terms ‘natural’ or ‘wild-type’ are used interchangeably herein when referring to HIV strains (or Env proteins therefrom), and refer to HIV strains (or Env proteins therefrom) as occurring in nature, e.g. such as in HIV-infected patients.

The invention generally relates to recombinant HIV envelope (Env) proteins comprising certain amino acid substitutions at indicated positions in the envelope protein sequence that stabilize the trimer form of the envelope protein. Introducing the identified amino acid substitution of the invention, and optionally one or more of the additional stabilizing mutations, into the sequence of an HIV envelope protein can result in an increased percentage of trimer formation and/or an increased trimer yield. This can for instance be measured using trimer-specific antibodies, size exclusion chromatography, and binding to antibodies that bind to correctly folded (stable trimeric) or alternatively to incorrectly folded (non-stable or non-trimeric) Env protein, and increased trimer percentage and/or trimer yield is considered indicative of stable, native, correctly folded Env protein.

Human immunodeficiency virus (HIV) is a member of the genus Lentivirinae, which is part of the family of Retroviridae. Two species of HIV infect humans: HIV-1 and HIV-2. HIV-1 is the most common strain of HIV virus, and is known to be more pathogenic than HIV-2. As used herein, the terms “human immunodeficiency virus” and “HIV” refer to, but are not limited to, HIV-1 and HIV-2. In preferred embodiments, HIV refers to HIV-1.

HIV is categorized into multiple clades with a high degree of genetic divergence. As used herein, the term “HIV clade” or “HIV subtype” refers to related human immunodeficiency viruses classified according to their degree of genetic similarity. The largest group of HIV-1 isolates is called Group M (major strains) and consists of at least twelve clades, A through L.

In one general aspect, the invention relates to a recombinant HIV envelope (Env) protein. The term “recombinant” when used with reference to a protein refers to a protein that is produced by a recombinant technique or by chemical synthesis in vitro. According to embodiments of the invention, a “recombinant” protein has an artificial amino acid sequence in that it contains at least one sequence element (e.g., amino acid substitution, deletion, addition, sequence replacement, etc.) that is not found in the corresponding naturally occurring sequence. Preferably, a “recombinant” protein is a non-naturally occurring HIV envelope protein that is optimized to induce an immune response or produce an immunity against one or more naturally occurring HIV strains.

The terms “HIV envelope protein,” “HIV Env,” and “HIV Env protein” refer to a protein, or a fragment or derivative thereof, that is in nature expressed on the envelope of the HIV virion and enables an HIV to target and attach to the plasma membrane of HIV infected cells. The terms “envelope” and “Env” are used interchangeably throughout the disclosure. The HIV env gene encodes the precursor protein gp160, which is proteolytically cleaved into the two mature envelope glycoproteins gp120 and gp41. The cleavage reaction is mediated by a host cell protease, furin (or by furin-like proteases), at a sequence motif highly conserved in retroviral envelope glycoprotein precursors. More specifically, gp160 trimerizes to (gp160)₃ and then undergoes cleavage into the two noncovalently associated mature glycoproteins gp120 and gp41. Viral entry is subsequently mediated by a trimer of gp120/gp41 heterodimers. Gp120 is the receptor binding fragment, and binds to the CD4 receptor (and the co-receptor) on a target cell that has such a receptor, such as, e.g., a T-helper cell. Gp41, which is non-covalently bound to gp120, is the fusion fragment and provides the second step by which HIV enters the cell. Gp41 is originally buried within the viral envelope, but when gp120 binds to a CD4 receptor and co-receptor, gp120 changes its conformation causing gp41 to become exposed, where it can assist in fusion with the host cell. Gp140 is the ectodomain of gp160.

According to embodiments of the invention, an “HIV envelope (Env) protein” can be a gp160 or gp140 protein, or combinations, fusions, truncations, or derivatives thereof. For example, an “HIV envelope protein” can include a gp120 protein noncovalently associated with a gp41 protein. An “HIV envelope protein” can also be a truncated HIV envelope protein including, but not limited to, envelope proteins comprising a C-terminal truncation in the ectodomain (i.e. the domain that extends into the extracellular space), a truncation in the gp41, such as a truncation in the ectodomain of gp41, in the transmembrane domain of gp41, or a truncation in the cytoplasmic domain of gp41. An HIV envelope protein can also be a gp140, corresponding to the gp160 ectodomain, or an extended or truncated version of gp140. Expression of gp140 proteins has been described in several publications (e.g. Zhang et al., 2001; Sanders et al., 2002; Harris et al., 2011), and the protein can also be ordered from service providers, in different variants e.g. based on different HIV strains. A gp140 protein according to the invention can have a cleavage site mutation so that the gp120 domain and gp41 ectodomain are not cleaved and covalently linked, or alternatively the gp120 domain and gp41 ectodomain can be cleaved and covalently linked, e.g. by a disulfide bridge (such as for instance in the SOSIP variants). An “HIV envelope protein” can further be a derivative of a naturally occurring HIV envelope protein having sequence mutations, e.g., in the furin cleavage sites, and/or so-called SOSIP mutations. An HIV envelope protein according to the invention can also have a cleavage site so that the gp120 and gp41 ectodomain can be non-covalently linked.

In preferred embodiments of the invention, the HIV Env protein is a gp140 protein or a gp160 protein, and more preferably a gp140 protein. In other preferred embodiments the Env protein is truncated, e.g. by deletion of the residues after the 7^{th} residue of the cytoplasmic region as compared to a natural Env protein.

According to embodiments of the invention, an “HIV envelope protein” can be a trimer or a monomer, and is preferably a trimer. The trimer can be a homotrimer (e.g., trimers comprising three identical polypeptide units) or a heterotrimer (e.g., trimers comprising three polypeptide units that are not all identical). Preferably, the trimer is a homotrimer. In case of a cleaved gp140 or gp160, it is a trimer of polypeptide units that are gp120-gp41 dimers, and in case all three of these dimers are the same, this is considered a homotrimer. In some cases the HIV envelope protein can also be present in the form of hexamers.

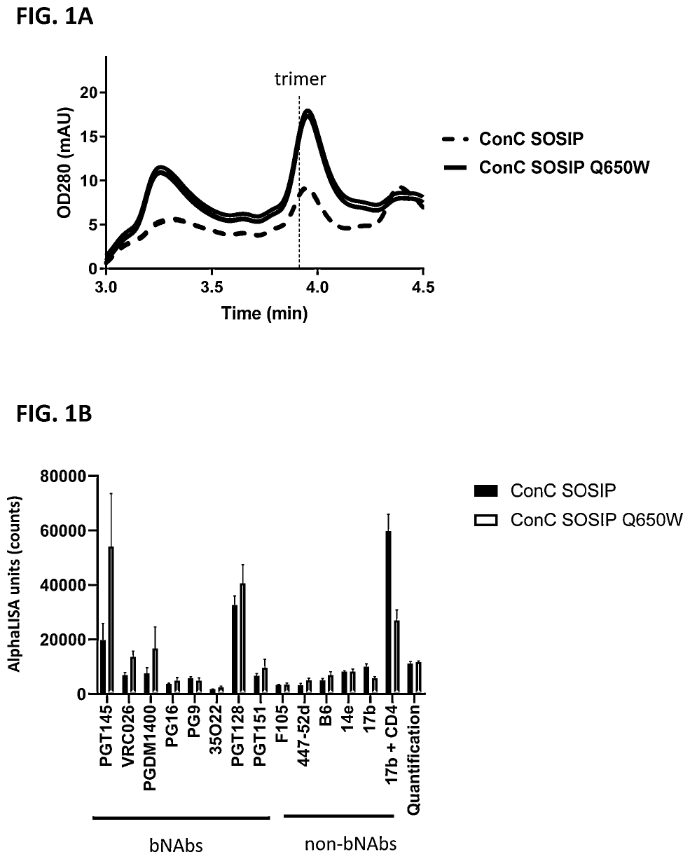

An “HIV envelope protein” can be a soluble protein, or a membrane bound protein. Membrane bound envelope proteins typically comprise a transmembrane domain, such as in the full length HIV envelope protein comprising a transmembrane domain (TM). Membrane bound proteins can have a cytoplasmic domain, but do not require a cytoplasmic domain to be membrane bound. Soluble envelope proteins comprise at least a partial or a complete deletion of the transmembrane domain. For instance, the C-terminal end of a full length HIV envelope protein can be truncated to delete the transmembrane domain, thereby producing a soluble protein (see e.g. FIGS. 1A and 1B in WO 2019/016062 for schematic representations of full length and truncated soluble HIV Env proteins, respectively). However, the HIV envelope protein can still be soluble with shorter truncations and alternative truncation positions to those shown in FIG. 1B of WO 2019/016062. Truncation can be done at various positions, and non-limiting examples are after amino acid 664, 655, 683, etc. which all result in soluble protein. A membrane-bound Env protein according to the invention may comprise a complete or a partial C-terminal domain (e.g. by partial deletion of the C-terminal cytoplasmic domain, e.g. in certain embodiments after the 7^{th} residue of the cytoplasmic region) as compared to a native Env protein. It will be clear to the skilled person that the deletion in the cytoplasmic region can also be from another than the 7^{th} residue of the cytoplasmic domain, e.g. after the 1^{st}, 2^{nd}, 3^{rd}, 4^{th}, 5^{th}, 6^{th}, 8^{th}, 9^{th}, 10^{th}, or any later residue of the cytoplasmic domain.

A signal peptide is typically present at the N-terminus of the HIV Env protein when expressed, but is cleaved off by signal peptidase and thus is not present in the mature protein. The signal peptide can be interchanged with other signal sequences, and two non-limiting examples of signal peptides are provided herein in SEQ ID NOs: 7 and 8.

According to embodiments of the invention, the HIV envelope protein, e.g., gp160, or gp140, can be derived from an HIV envelope protein sequence from any HIV clade (or ‘subtype’), e.g., clade A, clade B, clade C, clade D, clade E, clade F, clade G, clade H, etc, or combinations thereof (such as in ‘circulating recombinant forms’ or CRFs derived from recombination between viruses of different subtypes, e.g BC, AE, AG, BE, BF, ADG, etc). The HIV envelope protein sequence can be a naturally occurring sequence, a mosaic sequence, a consensus sequence, a synthetic sequence, or any derivative or fragment thereof. A “mosaic sequence” contains multiple epitopes derived from at least three HIV envelope sequences of one or more HIV clades and may be designed by algorithms that optimize the coverage of T-cell epitopes. Examples of sequences of mosaic HIV envelope proteins include those described in, e.g., Barouch et al, Nat Med 2010, 16: 319-323; WO 2010/059732; and WO 2017/102929. As used herein “consensus sequence” means an artificial sequence of amino acids based on an alignment of amino acid sequences of homologous proteins, e.g. as determined by an alignment (e.g. using Clustal Omega) of amino acid sequences of homologous proteins. It is the calculated order of most frequent amino acid residues, found at each position in a sequence alignment, based upon sequences of Env from for example at least 1000 natural HIV isolates. A “synthetic sequence” is a non-naturally occurring HIV envelope protein that is optimized to induce an immune response or produce immunity against more than one naturally occurring HIV strains. Mosaic HIV envelope proteins are non-limiting examples of synthetic HIV envelope proteins. In certain embodiments of the invention, the parent HIV Env protein is a consensus Env protein, or a synthetic Env protein. In the parent Env protein, a mutation is introduced to result in amino acid Trp, Phe, Met, or Leu, at position 650. In preferred embodiments, the mutation results in Trp or Phe at position 650 of the HIV Env protein. Optionally, such HIV Env protein may further have at least one of the indicated amino acids at the indicated positions (i)-(xx) described herein in Table 1. Particularly preferred are Env proteins having Trp, Phe, Met, or Leu, preferably Trp or Phe, at position 650, further having either (a) at least one, preferably at least two of the indicated amino acid residues at the indicated positions (i)-(viii), and/or (b) preferably having further SOSIP (e.g. indicated amino acids at position (xviii) and/or (c) furin cleavage site mutations (e.g. indicated amino acids at position (xvii), as described below.

In certain embodiments of the invention, an HIV envelope protein, whether a naturally occurring sequence, mosaic sequence, consensus sequence, synthetic sequence etc., comprises additional sequence mutations e.g., in the furin cleavage sites, and/or so-called SOSIP mutations.

In some embodiments of the invention, an HIV envelope protein of the invention has further mutations and is a “SOSIP mutant HIV Env protein.” The so-called SOSIP mutations are trimer stabilizing mutations that include the ‘SOS mutations’ (Cys residues at positions 501 and 605, which results in the introduction of a possible disulfide bridge between the newly created cysteine residues) and the ‘IP mutation’ (Pro residue at position 559). According to embodiments of the invention, a SOSIP mutant Env protein comprises at least one mutation selected from the group consisting of Cys at positions 501 and 605; Pro at position 559; and preferably Cys at positions 501 and 605 and Pro at position 559. A SOSIP mutant HIV Env protein can further comprise other sequence mutations, e.g., in the furin cleavage site. In addition, in certain embodiments it is possible to further add mutations such that the Env protein comprises Pro at position 556 or position 558 or at positions 556 and 558, which were found to be capable of acting not only as alternatives to Pro at position 559 in a SOSIP variant, but also as additional mutations that could further improve trimer formation of a SOSIP variant that already has Pro at position 559.

In certain preferred embodiments of the invention, a SOSIP mutant HIV Env protein comprises Cys at positions 501 and 605, and Pro at position 559.

In certain embodiments, an HIV envelope protein of the invention further comprises a mutation in the furin cleavage site. The mutation in the furin cleavage sequence can be an amino acid substitution, deletion, insertion, or replacement of one sequence with another, or replacement with a linker amino acid sequence. Preferably in the present invention, mutating the furin cleavage site can be used to optimize the cleavage site, so that furin cleavage is improved over wild-type, for instance by a replacement of the sequence at residues 508-511 with RRRRRR (SEQ ID NO: 6) [i.e. replacement of a typical amino acid sequence (e.g. EK) at positions 509-510 with four arginine residues (i.e. two replacements and two additions), while at positions 508 and 511, there are already arginine residues present in most HIV Env proteins, so these typically do not need to be replaced, but since the end result in literature is often referred to as amino acid sequence RRRRRR, we kept this nomenclature herein]. Other mutations that improve furin-cleavage are known and can also be used. Alternatively, it is possible to replace the furin cleavage site with a linker, so that furin cleavage is no longer necessary but the protein will adopt a native-like conformation (e.g. described in (Sharma et al, 2015) and (Georgiev et al, 2015)).

In particular embodiments of the invention, an HIV envelope protein of the invention further comprises both the so-called SOSIP mutations (preferably Cys at positions 501 and 605, and Pro at position 559) and a sequence mutation in the furin cleavage site, preferably a replacement of the sequence at residues 508-511 with RRRRRR (SEQ ID NO: 6). In certain preferred embodiments, the HIV Env comprises both the indicated SOSIP and furin cleavage site mutations, and in addition further comprises a Pro residue at position 556 or 558, most preferably at both positions 556 and 558.

In certain embodiments of the invention, the amino acid sequence of the HIV envelope protein is a consensus sequence, such as an HIV envelope clade C consensus or an HIV envelope Glade B consensus.

Exemplary HIV envelope proteins that can be used in the invention include HIV envelope Glade C consensus (SEQ ID NO: 2) and HIV envelope clade B consensus (SEQ ID NO: 4). These HIV envelope clade C and clade B consensus sequences can comprise additional mutations that, e.g., enhance stability and/or trimer formation, such as for instance the so-called SOSIP mutations and/or a sequence mutation in the furin cleavage site as described above, such as for instance in the ConC_SOSIP sequence shown in SEQ ID NO: 3 and the ConB_SOSIP sequence shown in SEQ ID NO: 5.

Other non-limiting examples of preferred HIV envelope protein sequences that can be used in the invention (as ‘background’ or ‘parent’ molecule, wherein then position 650 is mutated into Trp, Phe, Met, or Leu, preferably Trp or Phe) include synthetic HIV Env proteins, optionally having further SOSIP and/or furin cleavage site mutations as described above. Further non-limiting examples are mosaic HIV envelope proteins.

In certain embodiments, the parent molecule is a wild-type HIV Env protein. Such a parent molecule may optionally further have SOSIP and/or furin cleavage site mutations as described above.

Mutations resulting in the amino acid at position 650 being replaced with amino acid Trp, Phe, Met, or Leu, optionally further with the indicated amino acids at positions (i)-(xvii) described in Table 1, and/or optionally further comprising a mutation resulting in the amino acid at position 108 and/or 538 being replaced with amino acid His, can also be used in HIV Env proteins wherein no SOSIP mutations are present (e.g. in Env consensus sequences or in Env proteins from wild-type HIV isolates) and are likely to also improve the trimerization thereof, as the mutation of the invention is independent from the SOSIP mutations, having a different mode of action. Indeed, the additional stabilizing mutations for instance were shown to work in several different HIV Env protein backbones as described for instance in WO 2019/016062, including in the absence of the SOS-mutations as well as in the absence of the IP-mutation to improve HIV Env trimerization properties, as well as in the absence of any of the SOSIP mutations. Thus, in certain embodiments, an HIV Env protein according to the invention does not include any of the SOSIP mutations. In yet other embodiments, it is also possible to use alternatives for the SOSIP mutations to further stabilize the trimer. In certain alternative embodiments, a linker is used instead of the ‘SOS’ mutations. In certain alternative embodiments, instead of the ‘IP’ mutation one or both of positions 556 and/or 558 are replaced by a Pro residue.

A recombinant HIV envelope protein according to embodiments of the invention comprises an HIV envelope protein having certain amino acid residue(s) at specified positions in the amino acid sequence of an HIV envelope protein. In particular, it was shown that position 650 in the Env protein could be mutated to a Trp, Phe, Met, or Leu residue to improve trimer formation of the Env protein, wherein the numbering of the positions is according to the numbering in gp160 of HIV-1 isolate HXB2. In addition in optional embodiments, a number of positions in the envelope protein are indicated, as well as the particular amino acid residues to be desirable at one or more or each of the identified positions, in Table 1, wherein the numbering of the positions is according to the numbering in gp160 of HIV-1 isolate HXB2. An HIV Env protein according to the invention has Trp, Phe, Met, or Leu, preferably Trp or Phe at position 650, and optionally has the specified amino acid residue(s) in at least one of the indicated positions (i)-(xx) as provided in Table 1.

TABLE 1
Additional Desirable Amino Acids at Indicated Positions in the Recombinant
HIV Env Proteins According to Certain Embodiments

| No. | Position¹ | Desirable Amino Acid Residue |
|---|---|---|
| (i) | 651 | Phe, Leu, Met, or Trp (preferably Phe) |
| (ii) | 655 | Phe, Ile, Met, or Trp (preferably Ile) |
| (iii) | 535 | Asn or Gln (preferably Asn) |
| (iv) | 589 | Val, Ile, or Ala (preferably Val or Ile, most preferably Val) |
| (v) | 573 | Phe or Trp (preferably Phe) |
| (vi) | 204 | Ile |
| (vii) | 647 | Phe, Met, or Ile (preferably Phe) |
| (viii) | 658 | Val, Ile, Phe, Met, Ala, or Leu |
| | | (preferably Val or Ile, most preferably Val) |
| (ix) | 588 | Gln, Glu, Ile, Met, Val, Trp, or Phe (preferably Gln or Glu) |
| (x) | 64 or 66 | Lys at position 64; or Arg at position 66; |
| | | or Lys at position 64 and Arg at position 66 |
| (xi) | 316 | Trp |
| (xii) | 201 and 433 | Cys at both positions |
| (xiii) | 556 or 558 | Pro at either or both positions |
| | or 556 and | |
| | 558 | |
| (xiv) | 548-568 | Replacement by shorter and less flexible loop having 7-10 amino |
| | (HR1 loop) | acids, preferably a loop of 8 amino acids, e.g. having a sequence |
| | | chosen from any one of (SEQ ID NOs: 9-14) |
| (xv) | 568, 569, | Gly at any one of these positions, or Gly at both positions 568 |
| | 636 | and 636, or Gly at both positions 569 and 636 |
| (xvi) | 302, 519, | Tyr at position 302, or Arg at position 519, or Arg at position 520; or |
| | 520 | Tyr at position 302 and Arg at position 519; or Tyr at position 302 |
| | | and Arg at position 520; or Tyr at position 302 and Arg at both |
| | | positions 519 and 520 |
| (xvii) | 508-511 | Mutation at the furin cleavage site, preferably replacement at |
| | | positions 508-511 by RRRRRR (SEQ ID NO: 6) |
| (xviii) | 501 and | Cys at positions 501 and 605, or Pro at position 559, preferably Cys |
| | 605, or 559, | at positions 501 and 605 and Pro at position 559 |
| | or 501 and | |
| | 506 and 559 | |
| (xix) | 108 | His |
| (xx) | 538 | His |

¹According to the numbering in gp160 of HIV-1 isolate HXB2

The amino acid sequence of the HIV envelope protein into which the Trp, Phe, Met, or Leu at position 650, and optionally the one or more desirable amino acid (or indicated amino acid) substitutions at the one or more other indicated positions are introduced, is referred to as the “backbone HIV envelope sequence” or “parent HIV envelope sequence.” For example, if position 650 in the ConC_SOSIP sequence of SEQ ID NO: 3 is mutated to Trp, Phe, Met, or Leu, then the ConC_SOSIP sequence is considered to be the “backbone” or “parent” sequence. Any HIV envelope protein can be used as the “backbone” or “parent” sequence into which a novel stabilizing mutation (i.e. substitution of the amino acid at position 650 by Trp, Phe, Met, or Leu) according to an embodiment of the invention can be introduced, either alone or in combination with other mutations, such as the so-called SOSIP mutations and/or mutations in the furin cleavage site. Non-limiting examples of HIV Env protein that could be used as backbone include HIV Env protein from a natural HIV isolate, a synthetic HIV Env protein, or a consensus HIV Env protein.

According to certain embodiments of the invention, in addition to having Trp, Phe, Met, or Leu at position 650, the HIV envelope protein can optionally have the indicated amino acid residue at at least one of the indicated positions selected from the group consisting of positions (i)-(xx) in Table 1. Typically, it has been seen that HIV Env proteins comprising a combination of at least two, at least three, at least four, at least five, at least six, at least seven, etc of substitutions at the indicated positions (i)-(xviii), preferably including a combination of at least two, at least three, etc of substitutions at the indicated positions (i)-(viii), have improved trimerization properties as compared to backbone proteins not having or having less of such substitutions, see e.g. WO 2019/016062.

According to certain embodiments of the invention, in addition to having Trp, Phe, Met, or Leu, preferably Trp or Phe, at position 650, the HIV envelope protein can optionally also have His at position 108, or His at position 538, or His at both positions 108 and 538. These are other novel mutations that were shown to independently result in improved properties as shown herein. These positions are independent of each other and can in certain embodiments be combined to result in further improvement. Such molecules (having Trp, Phe, Met, or Leu at position 650 and His at position 538 and/or 108) may optionally further have the indicated amino acid residue at at least one of the indicated positions selected from the group consisting of positions (i)-(xviii) in Table 1.

Preferably, Trp, Phe, Met, or Leu at position 650, and/or at least one of the amino acids in (i)-(xx) is introduced into the recombinant HIV Env protein by amino acid substitution. For example, the recombinant HIV Env protein can be produced from an HIV Env protein that does not contain Trp, Phe, Met, or Leu at position 650 or that contains none or only one of the amino acid residues in (i)-(xx) above such that all or one or more of the indicated amino acid residues are introduced into the recombinant HIV Env protein by amino acid substitution. Likewise, His at position 108 and/or 538 can be introduced into the recombinant HIV Env protein by amino acid substitution.

The amino acid sequence of the HIV Env protein into which the above-described substitutions are introduced can be any HIV Env protein known in the art in view of the present disclosure, such as, for instance a naturally occurring sequence from HIV clade A, clade B, clade C, etc.; a mosaic sequence; a consensus sequence, e.g., clade B or clade C consensus sequence; a synthetic sequence; or any derivative or fragment thereof. In certain embodiments of the invention, the amino acid sequence of the HIV Env protein comprises additional mutations, such as, for instance, the so-called SOSIP mutations, and/or a mutation in the furin cleavage site.

In one particular embodiment, the HIV Env backbone protein is a SOSIP mutant HIV Env protein comprising at least one mutation selected from the group consisting of Cys at positions 501 and 605; Pro at position 559. In a preferred embodiment, the SOSIP mutant HIV Env protein comprises Cys at positions 501 and 605, and Pro at position 559. According to this embodiment, a recombinant HIV Env protein comprises the amino acid sequence of the SOSIP mutant HIV Env protein and an amino acid substitution at position 650 resulting in Trp, Phe, Met, or Leu at this position, and optionally one or more further amino acid substitutions by the indicated amino acid residue at at least one of the indicated positions selected from the group consisting of entries (i)-(xvi) in Table 1.

The SOSIP mutant HIV Env protein can further comprise a mutation in the furin cleavage site, such as a replacement at positions 608-511 by SEQ ID NO: 6.

In one particular embodiment, the HIV Env backbone protein is an HIV Env consensus Glade C comprising an amino acid sequence that is at least 95%, 96%, 97%, 98%, 99% or 100% identical to the amino acid sequence of SEQ ID NO: 2. In certain embodiments, the HIV consensus clade C sequence of SEQ ID NO: 2 further comprises the so-called SOSIP mutations, i.e., Cys at positions 501 and 605, and Pro at position 559, and in certain embodiments further comprises the so-called SOSIP mutations and a mutation in the furin cleavage site, such as for instance a replacement at positions 508-511 by SEQ ID NO: 6. In a particular embodiment, the HIV Env backbone protein comprises the sequence shown in SEQ ID NO: 3, or a sequence at least 95% identical thereto, wherein amino acids at positions 501, 559, 605, and 508-511 as replaced by SEQ ID NO: 6, are not mutated as compared to SEQ ID NO: 3.

In another particular embodiment, the HIV Env backbone protein is an HIV Env consensus clade B comprising an amino acid sequence that is at least 95%, 96%, 97%, 98%, 99% or 100% identical to the amino acid sequence of SEQ ID NO: 4. In certain embodiments, the HIV consensus clade B sequence of SEQ ID NO: 4 further comprises the so-called SOSIP mutations, i.e., Cys at positions 501 and 605, and Pro at position 559, and in certain embodiments further comprises the so-called SOSIP mutations and a mutation in the furin cleavage site, such as for instance a replacement at positions 508-511 by SEQ ID NO: 6. In a particular embodiment, the HIV Env backbone protein comprises the sequence shown in SEQ ID NO: 5, or a sequence at least 95% identical thereto, wherein amino acids at positions 501, 559, 605, and 508-511 as replaced by SEQ ID NO: 6, are not mutated as compared to SEQ ID NO: 5.

In yet another particular embodiment, the HIV Env backbone protein is a synthetic HIV Env protein, which may optionally have further SOSIP (501C, 605C, 559P) and/or furin cleavage site mutations (508-511RRRRRR) as described above.

In yet other particular embodiments, the HIV Env backbone protein is a HIV Env protein from a wild-type clade A, clade B, or clade C HIV virus, optionally comprising additional mutations to repair and/or stabilize the sequence according to methods described in WO 2018/050747 and WO 2019/016062.

In certain embodiments of the invention, a recombinant HIV Env protein according to the invention (i.e., having Trp, Phe, Met, or Leu at position 650, and optionally one or more indicated amino acid at positions (i)-(viii) in Table 1 above) can further comprise an indicated amino acid residue (e.g. via substitution) at one or more additional indicated positions selected from the group consisting of positions (ix)-(xvi) in Table 1. The amino acid substitutions were described previously, e.g. in WO 2019/016062. Certain of these amino acid substitutions (e.g. (ix)) were found to combine very well with (combinations of) mutations (i)-(viii), see e.g. WO 2019/016062. However, to the best of the knowledge of the inventors, these previously described mutations were not described in combination with the novel substitution described herein, i.e. Trp, Phe, Met, or Leu at position 650. These amino acid mutations in combination with the amino acid substitution of the invention can further increase trimer yield and/or the percentage of trimer formation. These amino acid substitutions can be introduced into any of the recombinant HIV Env proteins described herein in addition to substitution by the Trp, Phe, Met, or Leu amino acid residue at position 650, and optionally having further substitutions by the indicated amino acid residue at one or more of the indicated positions as described in Table 1 and/or His at position 108 and/or 538. The substitution identified in the present invention [W, F, M, or L at position 650; and likewise for H at position 538 and for H at position 108] is to the best of the inventors knowledge not present in natural (group M, i.e. overall) HIV Env sequences, is not found in combination with any of the substitutions (i)-(xx) of Table 1 in previously reported HIV Env protein sequences, and was not previously suggested to result in improved trimerization of the HIV Env protein, improved trimer yield and/or increased trimer stability. Clearly, the previously described mutations did not provide any suggestion for introduction of the mutation of the present invention, let alone the surprising effects thereof on trimer formation with a closed apex as for instance measured by antibody PGT145 binding. Apart from the point mutations (ix)-(xiii) in Table 1, it is also possible to replace the HR1 loop of the Env protein (amino acid residues 548-568 in a wild-type sequence, with numbering according to gp160 of the HXB2 isolate) by a shorter and less flexible loop having 7-10 amino acids, preferably a loop of 8 amino acids, e.g. having a sequence chosen from any one of (SEQ ID NOs: 9-14), see e.g. Kong et al (Nat Commun. 2016 June 28; 7:12040. doi: 10.1038/ncomms12040) that describes such shorter loops replacing the HR1 loop. Such an Env variant, further having the Trp, Phe, Met, or Leu amino acid residue at position 650, and optionally the indicated amino acid residues at at least one of the indicated positions (i)-(viii), is also an embodiment of the invention. Mutations listed in (ix)-(xiv) can in certain embodiments of the invention be added to HIV Env proteins of the invention, i.e. having Trp, Phe, Met, or Leu at position 650. In further embodiments these can be combined with mutations into one or more of the indicated amino acids at positions (i)-(viii). Also, combinations within the groups (ix)-(xiv) can be made. Again, any of those embodiments can be in any HIV Env protein, e.g. a wild-type isolate, a consensus Env, a synthetic Env protein, a SOSIP mutant Env protein, etc.

In certain embodiments, the HIV Env protein comprises a sequence that is at least 95% identical to, for example at least 96%, 97%, 98%, 99% identical to, or 100% identical to, any one of SEQ ID NOs: 2-5. For determination of the % identity, preferably the position 650, and preferably in addition the positions (i)-(xvi) of Table 1, and preferably also positions 108, 501, 538, 559 and 605 are not taken into account. It was found that Trp, Phe, Met, or Leu, preferably Trp or Phe at position 650 increased trimer percentage and trimer yield of the Env protein.

According to embodiments of the invention, a recombinant HIV Env protein has at least one of (a) an improved percentage of trimer formation, and (b) an improved trimer yield, compared to an HIV Env protein not having Trp, Phe, Met, or Leu at position 650 while further being identical (preferably compared to an HIV Env protein that has Gln at position 650 while further being identical).

As used herein “improved percentage of trimer formation” means that a greater percentage of trimer is formed when the backbone sequence of the HIV envelope protein contains Trp, Phe, Met, or Leu, preferably Trp or Phe at position 650 as compared to the percentage of trimer that is formed when the backbone sequence of the HIV envelope sequence contains a Gln residue at position 650 (Gln is the amino acid present in the majority of natural Glade C variants of HIV-1 Env at this position). More generally, “improved percentage of trimer formation” means that a greater percentage of trimer is formed when the backbone sequence of the HIV envelope protein contains substitution of the amino acid at position 650 into Trp, Phe, Met, or Leu, preferably Trp or Phe, and optionally one or more of the amino acids substitutions described in Table 1 as compared to the percentage of trimer that is formed when the backbone sequence of the HIV envelope sequence does not contain such amino acid substitutions. As used herein “improved trimer yield” means that a greater total amount of the trimer form of the envelope protein is obtained when the backbone sequence of the HIV envelope protein contains Trp, Phe, Met, or Leu, preferably Trp or Phe at position 650 as compared to the total amount of trimer form of the envelope protein that is obtained when the backbone sequence of the HIV envelope sequence contains a Gln residue at position 650. More generally, “improved trimer yield” means that a greater total amount of the trimer form of the envelope protein is obtained when the backbone sequence of the HIV envelope protein contains one or more of the amino acid substitutions described in Table 1 as compared to the total amount of trimer form of the envelope protein that is obtained when the backbone sequence of the HIV envelope sequence does not contain such amino acid substitutions.

Trimer formation can be measured by an antibody binding assay using antibodies that bind specifically to the trimer form of the HIV Env protein. Examples of trimer specific antibodies that can be used to detect the trimer form include, but are not limited to, the monoclonal antibodies (mAbs) PGT145, PGDM1400, PG16, and PGT151. Preferably, the trimer specific antibody is mAb PGT145. Any antibody binding assay known in the art in view of the present disclosure can be used to measure the percentage of trimer formation of a recombinant HIV Env protein of the invention, such as ELISA, AlphaLISA, etc.

In a particular embodiment, trimer formation is measured by AlphaLISA. AlphaLISA is a bead-based proximity assay in which singlet oxygen molecules, generated by high energy irradiation of donor beads, are transferred to acceptor beads that are within a distance of approximately 200 nm with respect to the donor beads. The transfer of singlet oxygen molecules to the acceptor beads initiates a cascading series of chemical reactions resulting in a chemiluminescent signal that can then be detected (Eglen et al. Curr. Chem. Genomics, 2008, 25(1): 2-10). For example, recombinant HIV envelope proteins labeled with a Flag-His tag can be incubated with a trimer specific mAb, donor beads conjugated to the antibody that binds to the trimer specific mAb, nickel-conjugated donor beads, acceptor beads conjugated to an anti-His antibody, and acceptor beads conjugated to an anti-Flag antibody. The amount of trimer formed can be determined by measuring the chemiluminescent signal generated from the pair of donor beads conjugated to the antibody that binds to the trimer specific mAb and the acceptor beads conjugated to the anti-His antibody. The total amount of HIV envelope protein expressed can be determined by measuring the chemiluminescent signal generated from the pair of nickel-conjugated donor beads and anti-Flag-conjugated acceptor beads. For example, the amount of trimer and the total envelope protein expressed can be measured by an AlphaLISA assay as described in detail in Example 3 of WO 2019/016062. The percentage of trimer formation can be calculated by dividing the amount of trimer formed by the total amount of expressed envelope protein. In certain embodiments, the trimer formation is measured by binding to broadly neutralizing HIV Env binding antibody PGT145, PGDM1400, or both, and compared under the same conditions (e.g. in an AlphaLISA assay) to such binding to a parent molecule not having the mutation of the invention (each of such antibodies is available to the skilled person, as it has been previously described (see e.g. Lee et al, 2017, Immunity 46: 690-702, including supplemental information) and is available from various sources such as the NIH AIDS reagent program, or from Creative Biolabs, or can be recombinantly produced based upon their known sequence; other useful antibodies described herein are also known from the prior art and can be obtained by similar means). In certain embodiments, the binding to antibodies PGT145 and/or PGDM1400 is increased for a HIV Env protein of the invention as compared to a HIV Env parent protein, and in certain embodiments the binding to non-broadly neutralizing antibody 17b is about the same or preferably reduced for a HIV Env protein of the invention as compared to a HIV Env parent protein.

The amount of trimer formed and the total amount of envelope protein expressed can also be determined using chromatographic techniques that are capable of separating the trimer form from other forms of the HIV envelope protein, e.g., the monomer form. Examples of such techniques that can be used include, but are not limited to size exclusion chromatography (SEC), e.g. analytical SEC, or SEC multi-angle light scattering (SEC-MALS). According to certain embodiments, the percentage of trimer formation is determined using SEC-MALS or (analytical) SEC. According to certain embodiments, the trimer yield is determined using SEC-MALS or (analytical) SEC.

The invention in certain embodiments also provides a method for improving the trimer formation of an HIV Env protein, the method comprising substituting the residue at position 650 (typically Gln) of a parent HIV Env protein with Trp, Phe, Met, or Leu, preferably with Trp or Phe. This can for instance be done using standard molecular biology technology.

Nucleic Acid, Vectors, and Cells

In another general aspect, the invention provides a nucleic acid molecule encoding a recombinant HIV Env protein according to the invention, and a vector comprising the nucleic acid molecule. The nucleic acid molecules of the invention can be in the form of RNA or in the form of DNA obtained by cloning or produced synthetically. The DNA can be double-stranded or single-stranded. The DNA can for example comprise cDNA, genomic DNA, or combinations thereof. The nucleic acid molecules and vectors can be used for recombinant protein production, expression of the protein in a host cell, or the production of viral particles.

In certain embodiments, the nucleic acid molecules encoding the proteins according to the invention are codon-optimized for expression in mammalian cells, preferably human cells, or insect cells. Methods of codon-optimization are known and have been described previously (e.g. WO 96/09378 for mammalian cells). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

It will be understood by a skilled person that numerous different polynucleotides and nucleic acid molecules can encode the same protein as a result of the degeneracy of the genetic code. It is also understood that skilled persons may, using routine techniques, make nucleotide substitutions that do not affect the protein sequence encoded by the nucleic acid molecules to reflect the codon usage of any particular host organism in which the proteins are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may or may not include introns.

Nucleic acid sequences can be cloned using routine molecular biology techniques, or generated de novo by DNA synthesis, which can be performed using routine procedures by service companies having business in the field of DNA and/or RNA synthesis and/or molecular cloning.

Nucleic acid encoding the recombinant HIV Env protein of the invention can for instance also be in the form of mRNA. Such mRNA can be directly used to produce the Env protein, e.g. in cell culture, but also via vaccination, e.g. by administering the mRNA in a drug delivery vehicle such as liposomes or lipid nanoparticles. The nucleic acid or mRNA may also be in the form of self-amplifying RNA or self-replicating RNA, e.g. based on the self-replicating mechanism of positive-sense RNA viruses such as alphaviruses. Such self-replicating RNA (or repRNA or RNA replicon) may be in the form of an RNA molecule expressing alphavirus nonstructural protein genes such that it can direct its own replication amplification in a cell, without producing a progeny virus. For example, a repRNA can comprise 5′ and 3′ alphavirus replication recognition sequences, coding sequences for alphavirus nonstructural proteins, a heterologous gene encoding an antigen, such as the HIV Env protein of the invention, and the means for expressing the antigen, and a polyadenylation tract. Such repRNAs induce transient, high-level antigen expression in a broad range of tissues within a host, and are able to act in both dividing and non-dividing cells. RepRNAs can be delivered to a cell as a DNA molecule, from which a repRNA is launched, packaged in a viral replicon particle (VRP), or as a naked modified or unmodified RNA molecule. In certain embodiments, the mRNA may be nucleoside-modified, e,g, an mRNA or replicating RNA can contain modified nucleobases, such as those described in US2011/0300205. A non-limiting example of repRNA can be found in WO 2019/023566. In non-limiting embodiments, mRNA vaccines and self-amplifying RNA vaccines can for instance include vaccine formats and variations as described in (Pardi et al, 2018, Nature Reviews Drug Discovery 17: 261-279) and in (Zhang et al, 2019, Front. Immunol. 10: 594).

According to embodiments of the invention, the nucleic acid encoding the recombinant HIV envelope protein is operably linked to a promoter, meaning that the nucleic acid is under the control of a promoter. The promoter can be a homologous promoter (i.e., derived from the same genetic source as the vector) or a heterologous promoter (i.e., derived from a different vector or genetic source). Non-limiting examples of suitable promoters include the human cytomegalovirus immediate early (hCMV IE, or shortly “CMV”) promoter and the Rous Sarcoma virus (RSV) promoter. Preferably, the promoter is located upstream of the nucleic acid within an expression cassette.

The nucleic acid according to the invention may be incorporated into a vector. In certain embodiments a vector comprises DNA and/or RNA. According to embodiments of the invention, a vector can be an expression vector. Expression vectors include, but are not limited to, vectors for recombinant protein expression and vectors for delivery of nucleic acid into a subject for expression in a tissue of the subject, such as a viral vector. Examples of viral vectors suitable for use with the invention include, but are not limited to adenoviral vectors, adeno-associated virus vectors, pox virus vectors, Modified Vaccinia Ankara (MVA) vectors, enteric virus vectors, Venezuelan Equine Encephalitis virus vectors, Semliki Forest Virus vectors, Tobacco Mosaic Virus vectors, lentiviral vectors, alphavirus vectors, etc. The vector can also be a non-viral vector. Examples of non-viral vectors include, but are not limited to plasmids, bacterial artificial chromosomes, yeast artificial chromosomes, bacteriophages, etc.

In certain embodiments of the invention, the vector is an adenovirus vector, e.g., a recombinant adenovirus vector. A recombinant adenovirus vector may for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. In other embodiments, the recombinant adenovirus is based upon a chimpanzee adenovirus such as ChAdOx 1 (see e.g. WO 2012/172277), or ChAdOx 2 (see e.g. WO 2018/215766), or BZ28 (see e.g. WO 2019/086466). In other embodiments, the recombinant adenovirus is based upon a gorilla adenovirus such as BLY6 (see e.g. WO 2019/086456), or BZ1 (see e.g. WO 2019/086466).

The preparation of recombinant adenoviral vectors is well known in the art. For example, preparation of recombinant adenovirus 26 vectors is described, in, e.g., WO 2007/104792 and in Abbink et al., (2007) Virol. 81(9): 4654-63. Exemplary genome sequences of adenovirus 26 are found in GenBank Accession EF 153474 and in SEQ ID NO: 1 of WO 2007/104792. Exemplary genome sequences for rhAd51, rhAd52 and rhAd53 are provided in US 2015/0291935.

According to embodiments of the invention, any of the recombinant HIV Env proteins described herein can be expressed and/or encoded by any of the vectors described herein. In view of the degeneracy of the genetic code, the skilled person is well aware that several nucleic acid sequences can be designed that encode the same protein, according to methods entirely routine in the art. The nucleic acid encoding the recombinant HIV Env protein of the invention can optionally be codon-optimized to ensure proper expression in the host cell (e.g., bacterial or mammalian cells). Codon-optimization is a technology widely applied in the art.

The invention also provides cells, preferably isolated cells, comprising any of the nucleic acid molecules and vectors described herein. The cells can for instance be used for recombinant protein production, or for the production of viral particles.

Embodiments of the invention thus also relate to a method of making a recombinant HIV Env protein. The method comprises transfecting a host cell with an expression vector comprising nucleic acid encoding a recombinant HIV Env protein according to an embodiment of the invention operably linked to a promoter, growing the transfected cell under conditions suitable for expression of the recombinant HIV Env protein, and optionally purifying or isolating the recombinant HIV Env protein expressed in the cell. The recombinant HIV Env protein can be isolated or collected from the cell by any method known in the art including affinity chromatography, size exclusion chromatography, etc. Techniques used for recombinant protein expression will be well known to one of ordinary skill in the art in view of the present disclosure. The expressed recombinant HIV Env protein can also be studied without purifying or isolating the expressed protein, e.g., by analyzing the supernatant of cells transfected with an expression vector encoding the recombinant HIV Env protein and grown under conditions suitable for expression of the HIV Env protein.

In a preferred embodiment, the expressed recombinant HIV Env protein is purified under conditions that permit association of the protein so as to form the stabilized trimeric complex. For example, mammalian cells transfected with an expression vector encoding the recombinant HIV Env protein operably linked to a promoter (e.g. CMV promoter) can be cultured at 33-39° C., e.g. 37° C., and 2-12% CO₂, e.g. 8% CO₂. Expression can also be performed in alternative expression systems such as insect cells or yeast cells, all conventional in the art. The expressed HIV Env protein can then be isolated from the cell culture for instance by lectin affinity chromatography, which binds glycoproteins. The HIV Env protein bound to the column can be eluted with mannopyranoside. The HIV Env protein eluted from the column can be subjected to further purification steps, such as size exclusion chromatography, as needed, to remove any residual contaminants, e.g., cellular contaminants, but also Env aggregates, gp140 monomers and gp120 monomers. Alternative purification methods, non-limiting examples including antibody affinity chromatography, negative selection with non-bNAbs, anti-tag purification, or other chromatography methods such as ion exchange chromatography etc, as well as other methods known in the art, could also be used to isolate the expressed HIV Env protein.

The nucleic acid molecules and expression vectors encoding the recombinant HIV Env proteins of the invention can be made by any method known in the art in view of the present disclosure. For example, nucleic acid encoding the recombinant HIV Env protein can be prepared by introducing mutations that encode the one or more amino acid substitutions at the indicated positions into the backbone HIV envelope sequence using genetic engineering technology and molecular biology techniques, e.g., site directed mutagenesis, polymerase chain reaction (PCR), etc., which are well known to those skilled in the art. The nucleic acid molecule can then be introduced or “cloned” into an expression vector also using standard molecular biology techniques. The recombinant HIV envelope protein can then be expressed from the expression vector in a host cell, and the expressed protein purified from the cell culture by any method known in the art in view of the present disclosure.

Trimeric Complex

In another general aspect, the invention relates to a trimeric complex comprising a noncovalent oligomer of three of the recombinant HIV Env proteins according to the invention. The trimeric complex can comprise any of the recombinant HIV Env proteins described herein. Preferably the trimeric complex comprises three identical monomers (or identical heterodimers if gp140 is cleaved) of the recombinant HIV Env proteins according to the invention. The trimeric complex can be separated from other forms of the HIV envelope protein, such as the monomer form, or the trimeric complex can be present together with other forms of the HIV envelope protein, such as the monomer form.

Compositions and Methods

In another general aspect, the invention relates to a composition comprising a recombinant HIV Env protein, trimeric complex, isolated nucleic acid, vector, or host cell, and a pharmaceutically acceptable carrier. The composition can comprise any of the recombinant HIV Env proteins, trimeric complexes, isolated nucleic acid molecules, vectors, or host cells described herein.

A carrier can include one or more pharmaceutically acceptable excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions of the invention can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the invention can also be formulated for other routes of administration including transmucosal, ocular, rectal, long acting implantation, sublingual administration, under the tongue, from oral mucosa bypassing the portal circulation, inhalation, or intranasal.

Embodiments of the invention also relate to methods of making the composition. According to embodiments of the invention, a method of producing a composition comprises mixing a recombinant HIV Env protein, trimeric complex, isolated nucleic acid, vector, or host cell of the invention with one or more pharmaceutically acceptable carriers. One of ordinary skill in the art will be familiar with conventional techniques used to prepare such compositions.

HIV antigens (e.g., proteins or fragments thereof derived from HIV gag, pol, and/or env gene products) and vectors, such as viral vectors, expressing the HIV antigens have previously been used in immunogenic compositions and vaccines for vaccinating a subject against an HIV infection, or for generating an immune response against an HIV infection in a subject. As used herein, “subject” means any animal, preferably a mammal, most preferably a human, to who will be or has been administered an immunogenic composition according to embodiments of the invention. The term “mammal” as used herein, encompasses any mammal. Examples of mammals include, but are not limited to, mice, rats, rabbits, guinea pigs, monkeys, humans, etc., preferably a human. The recombinant HIV Env proteins of the invention can also be used as antigens to induce an immune response against human immunodeficiency virus (HIV) in a subject in need thereof. The immune response can be against one or more HIV clades, such as Glade A, clade B, clade C, etc. The compositions can comprise a vector from which the recombinant HIV Env protein is expressed, or the composition can comprise an isolated recombinant HIV Env protein according to an embodiment of the invention.

For example, compositions comprising a recombinant HIV protein or a trimeric complex thereof can be administered to a subject in need thereof to induce an immune response against an HIV infection in the subject. A composition comprising a vector, such as an adenovirus vector, encoding a recombinant HIV Env protein of the invention, wherein the recombinant HIV Env protein is expressed by the vector, can also be administered to a subject in need thereof to induce an immune response against an HIV infection in the subject. The methods described herein also include administering a composition of the invention in combination with one or more additional HIV antigens (e.g., proteins or fragments thereof derived from HIV gag, pol, and/or env gene products) that are preferably expressed from one or more vectors, such as adenovirus vectors or MVA vectors, including methods of priming and boosting an immune response.

In certain embodiments, the HIV Env protein can be displayed on a particle, such as a liposome, virus-like particle (VLP), nanoparticle, virosome, or exosome, optionally in combination with endogenous and/or exogenous adjuvants. When compared to soluble or monomeric Env protein on its own, such particles typically display enhanced efficacy of antigen presentation in vivo.

Examples of VLPs that display HIV Env protein can be prepared e.g. by co-expressing the HIV Env protein with self-assembling viral proteins such as HIV Gag core or other retroviral Gag proteins. VLPs resemble viruses, but are non-infectious because they contain no viral genetic material. The expression of viral structural proteins, such as envelope or capsid, can result in self-assembly of VLPs. VLPs are well known to the skilled person, and their use in vaccines is for instance described in (Kushnir et al, 2012).

In certain preferred embodiments, the particle is a liposome. A liposome is a spherical vesicle having at least one lipid bilayer. The HIV Env trimer proteins can for instance be non-covalently coupled to such liposomes by electrostatic interactions, e.g. by adding a His-tag to the C-terminus of the HIV Env trimer and a bivalent chelating atom such as Ni²⁺ or Co²⁺ incorporated into the head group of derivatized lipids in the liposome. In certain non-limiting and exemplary embodiments, the liposome comprises 1,2-distearoyl-sn-glycero-3-phosphocholine (DSPC), cholesterol, and the Nickel or Cobalt salt of 1,2-dioleoyl-sn-glycero-3-[(N-(5-amino-1-carboxypentyl)iminodiacetic acid)succinyl] (DGS-NTA(Ni²⁺) or DGS-NTA(Co²⁺)) at 60:36:4 molar ratio. In preferred embodiments, the HIV Env trimer proteins are covalently coupled to the liposomal surface, e.g. via a maleimide functional group integrated in the liposome surface. In certain non-limiting exemplary embodiments thereof, the liposome comprises DSPC, cholesterol, and 1,2-dipalmitoyl-sn-glycero-3-phosphoethanolamine-N-[4-(p-maleimidomethyl)cyclohexane-carboxamide] lipid in a molar ratio of 54:30:16. The HIV Env protein can be coupled thereto e.g. via an added C-terminal cysteine in the HIV Env protein. The covalently coupled variants are more stable, elicit high antigen specific IgG titers and epitopes at the antigenically less relevant ‘bottom’ of the Env trimer are masked. Methods for preparing HIV Env trimers coupled to liposomes, as well as their characterization, are known and have for instance been described in (Bale et al, 2017), incorporated by reference herein. The invention also provides an HIV Env protein of the invention fused to and/or displayed on a liposome.

In certain embodiments, a HIV Env protein of the invention is fused to self-assembling particles, or displayed on nanoparticles. Antigen nanoparticles are assemblies of polypeptides that present multiple copies of antigens, e.g. the HIV Env protein of the instant invention, which result in multiple binding sites (avidity) and can provide improved antigen stability and immunogenicity. Preparation and use of self-assembling protein nanoparticles for use in vaccines is well-known to the skilled person, see e.g. (Zhao et al, 2014), (López-Sagaseta et al, 2016). As non-limiting examples, self-assembling nanoparticles can be based on ferritin, bacterioferritin, or DPS. DPS nanoparticles displaying proteins on their surface are for instance described in WO2011/082087. Description of trimeric HIV-1 antigens on such particles has for instance been described in (He et al, 2016). Other self-assembling protein nanoparticles as well as preparation thereof, are for instance disclosed in WO 2014/124301, and US 2016/0122392, incorporated by reference herein. The invention also provides an HIV Env protein of the invention fused to and/or displayed on a self-assembling nanoparticle. The invention also provides compositions comprising VLPs, liposomes, or self-assembling nanoparticles according to the invention.

In certain embodiments, an adjuvant is included in a composition of the invention or co-administered with a composition of the invention. Use of adjuvant is optional, and may further enhance immune responses when the composition is used for vaccination purposes. Adjuvants suitable for co-administration or inclusion in compositions in accordance with the invention should preferably be ones that are potentially safe, well tolerated and effective in people. Such adjuvants are well known to the skilled person, and non-limiting examples include QS-21, Detox-PC, MPL-SE, MoGM-CSF, TiterMax-G, CRL-1005, GERBU, TERamide, PSC97B, Adjumer, PG-026, GSK-I, GcMAF, B-alethine, MPC-026, Adjuvax, CpG ODN, Betafectin, Aluminium salts such as Aluminium Phosphate (e.g. AdjuPhos) or Aluminium Hydroxide, and MF59.

Also disclosed herein are recombinant HIV envelope proteins comprising an amino acid sequence that is at least 95%, 96%, 97%, 98%, 99% or 100% identical to the amino acid sequence of SEQ ID NO: 2 or SEQ ID NO: 4, which represent the HIV envelope consensus Glade C and consensus clade B sequences, respectively. A recombinant HIV envelope protein comprising an amino acid sequence that is at least 95%, 96%, 97%, 98%, 99% or 100% identical to the amino acid sequence of SEQ ID NO: 2 or SEQ ID NO: 4 can optionally further comprise the so-called SOSIP mutations and/or a mutation in the furin cleavage site, such as, for instance in those sequences shown in SEQ ID NO: 3, or SEQ ID NO: 3 further comprising Pro at position 558 and/or position 556; and SEQ ID NO: 5, or SEQ ID NO: 5 further comprising Pro at position 558 and/or position 556. When determining the % identity for these sequences, the amino acids at the mutated furin cleavage site and at positions 501, 605, 559, 556 and 558 are preferably not taken into account. Such proteins are expressed at high levels and have a high level of stability and trimer formation. Such HIV Env proteins can in certain embodiments be used as backbone proteins, wherein the mutation of T538 into H can be made to obtain a molecule of the invention. Isolated nucleic acid molecules encoding these sequences, vectors comprising these sequences operably linked to a promoter, and compositions comprising the protein, isolated nucleic acid molecule, or vector are also disclosed.

### EXAMPLES

### Example 1: Mutation of HIV Envelope at Position 650 into Trp, Phe, Met, or Leu Increases the Trimer Yield

HIV clade C and clade B envelope (Env) protein consensus sequences including SOSIP mutations (cysteine residues at positions 501 and 605 and a proline residue at position 559) as well as optimized furin cleavage site by replacing the furin site at residues 508-511 with 6 arginine residues were used as the backbone sequence for studying the effects of a mutation at position 650 on trimer formation of the HIV Env proteins. In addition, the C-terminus was truncated at residue 664, resulting in a sequence encoding a soluble HIV gp140 protein. Further, Val at position 295 was mutated into an Asn (V295N) in the clade C variant (ConC_SOSIP), to create an N-linked glycosylation site present in the majority of HIV strains and that can improve binding to certain antibodies used in some experiments. All positions of substitution/modification described above are relative to the numbering in gp160 of HIV-1 isolate HXB2. Backbone clade C and clade B HIV gp140 sequences, referred to as “ConC_SOSIP,” and “ConB_SOSIP”, respectively, are shown in (SEQ ID NOs: 3 and 5). In particular, the Gln residue at position 650 was replaced by a Trp residue (Q650W mutation, also referred to as one of the ‘mutations of the invention’) in these backbone molecules. In addition, the Gln residue at position 650 was also replaced in the ConC_SOSIP backbone by Phe (Q650F), Met (Q650M), Ile (Q650I) or Leu (Q650L) residues, of which Q650F, Q650M, and Q650L are also referred to as ‘mutations of the invention’). Similarly, the Ile residue at position 108 was replaced by a His residue (I108H mutation) in the ConC_SOSIP and ConB_SOSIP backbones. Similarly, the Thr residue at position 538 was replaced by a His residue (T538H mutation) in the ConC_SOSIP and ConB_SOSIP backbones. The resulting recombinant HIV Env proteins were expressed as soluble gp140 proteins. The experiments were carried out according to known methods, e.g. as described in WO 2018/050747.

AlphaLISA Assay

AlphaLISA® (Perkin-Elmer) is a bead-based proximity assay in which singlet oxygen molecules generated by high energy irradiation of Donor beads transfers to Acceptor beads which are within a distance of approximately 200 nm. It is a sensitive high throughput screening assay that does not require washing steps. A cascading series of chemical reactions results in a chemiluminescent signal (Eglen et al. Curr Chem Genomics, 2008). For the AlphaLISA assay the constructs were equipped with a sortase A-Flag-His tag (SEQ ID NO: 15). The HIV constructs were expressed in Expi293F cells, which were cultured for 3 days in 96 well plates (200 μl/well). Crude supernatants were diluted 120 times in AlphaLISA® buffer (PBS+0.05% Tween-20+0.5 mg/mL BSA) except for 17b-based assays, in which supernatants were diluted 12 times. Subsequently 10 μl of these dilutions were transferred to a half-area 96-well plate and mixed with a 40 μl mix of acceptor beads, donor beads and mAb. The beads were mixed well before use. After 2 hours of incubation at RT, non-shaking, the signal was measured with Neo (BioTek) The donor beads were conjugated to ProtA (Cat #: AS102M, Perkin Elmer), which could bind to the mAb. The acceptor beads were conjugated to an anti-His antibody (Cat #: AL112R, Perkin Elmer) to detect the His-tag of the protein. For the quantification of the total protein level, a combination of Nickel-conjugated donor beads (Cat #: AS101M, Perkin Elmer) together with acceptor beads carrying anti-Flag antibody (Cat #: AL112R, Perkin Elmer) were used. For 17b in combination with sCD4-His, a combination of ProtA donor beads and anti-Flag acceptor beads was used. The average signal of mock transfections (no Env) was subtracted from the AlphaLISA counts measured for the different Env proteins. As a reference the parent ConC_SOSIP or ConB_SOSIP Env plasmids were used, respectively for the clade C and clade B Env mutants.

The monoclonal antibodies (mAbs) that were used for analysis are well known in the field (see e.g. WO 2018/050747), and are indicated in Table 2 with some of their features.

TABLE 2
HIV Env antibodies used in experiments

| | broadly | | trimer |
|---|---|---|---|
| mAb | neutralizing | epitope | specific |
| PGT145 | yes | apex | yes |
| VRC026 | yes | apex | yes |
| PGDM1400 | yes | apex | yes |
| PG16 | yes | apex | yes |
| PG9 | yes | apex | no |
| 35O22 | yes | gp120-gp41 interface | no |
| PGT128 | yes | V3 base | no |
| PGT151 | yes | gp120-gp41 interface | yes |
| F105 | no | CD4bs | no |
| 447-52d | no | V3 crown | no |
| B6 | no | CD4bs | no |
| 14e | no | V3 crown | no |
| 17b | no | CCR5bs | no |
| 17b + CD4 | NA | CD4bs & CCR5bs | no |
| Quantification | NA | tag | no |

The broadly neutralizing antibodies (bNAbs) bind the native prefusion conformation of Env from many HIV strains. The non-bNAbs bind either misfolded, non-native Envs or a highly variable exposed loop. Protein folding was also tested by measuring the binding of soluble HIV gp140 Env protein variants to an antibody (mAb 17b) known to bind the co-receptor binding site of the HIV envelope protein, which is exposed only after binding of CD4 (data not shown). In particular, soluble receptor CD4 (sCD4) was used in combination with mAb 17 to evaluate CD4-induced conformational change. Binding of mAb 17b to the HIV gp140 Env protein variant without prior CD4 binding to the envelope protein is an indication of partially unfolded or pre-triggered envelope protein (i.e., an unstable Env that adopts the “open” conformation in the absence of CD4 binding).

Generally, it is thus a positive attribute for HIV Env variants if binding of one or more bNAbs increases and binding of one or more non-bNAbs does not increase or even decreases, as compared to a parent Env molecule in these experiments.

Analytical SEC

The HIV Env variants were expressed in 96 well format cell cultures. An ultra-high-performance liquid chromatography system (Vanquish, Thermo Scientific) and μDAWN TREOS instrument (Wyatt) coupled to an Optilab μT-rEX Refractive Index Detector (Wyatt) in combination with an in-line Nanostar DLS reader (Wyatt) was used for performing the analytical size exclusion chromatography (analytical SEC) experiment. The cleared crude cell culture supernatants were applied to a TSK-Gel UP-SW3000 4.6×150 mm column with the corresponding guard column (Tosoh Bioscience) equilibrated in running buffer (150 mM sodium phosphate, 50 mM sodium chloride, pH 7.0) at 0.3 mL/min. When analyzing supernatant samples, μMALS detectors were offline and analytical SEC data was analyzed using Chromeleon 7.2.8.0 software package. The signal of supernatants of non-transfected cells was subtracted from the signal of supernatants of HIV Env transfected cells.

The recombinant HIV Env protein variants generated were screened for trimer formation to check whether the Q650W mutation improved the percentage of trimer formed and/or improved trimer yields relative to the backbone sequences. Analytical SEC (FIG. 1A, 2A) was used to determine trimer yield. An AlphaLISA assay to evaluate the binding of a panel of broadly neutralizing HIV antibodies (bNAbs) and non-bNAbs to the recombinant HIV Env proteins was used to verify relative trimer yields and to determine conformational characteristics of the HIV Env proteins (FIG. 1B, 2B).

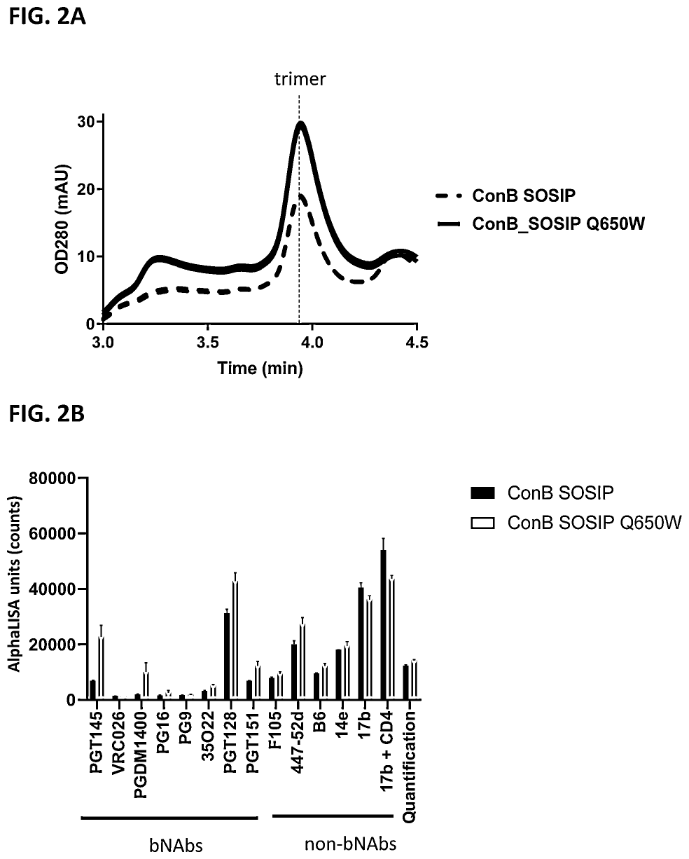

In analytical SEC, it was shown that the mutation Q650W increased trimer yield of both ConC_SOSIP and ConB_SOSIP (FIGS. 1A and 2A). Furthermore, the mutation Q650W increased bNAb antibody binding in AlphaLISA compared to its parent molecule not having the mutation. An increase in trimer-specific apex-directed broadly neutralizing antibodies (bNAbs) PGT145, VRCO26, and PGDM1400, was demonstrated, indicating improved trimer yield and/or trimer folding of ConC_SOSIP (FIG. 1B). The same observation was made for ConB_SOSIP, with the exception of VRCO26, which does not bind to this HIV Env irrespective of stabilization (FIG. 2B). Q650W reduces the binding of the non-bNAb 17b in AlphaLISA for both ConC_SOSIP and ConB_SOSIP (FIGS. 1B and 2B), which is a desired characteristic and indicates a closed native prefusion conformation of the Env trimer. The increased binding to mAb 17b in the presence of CD4 demonstrates that the epitope for this non-bNAb 17b is still intact.

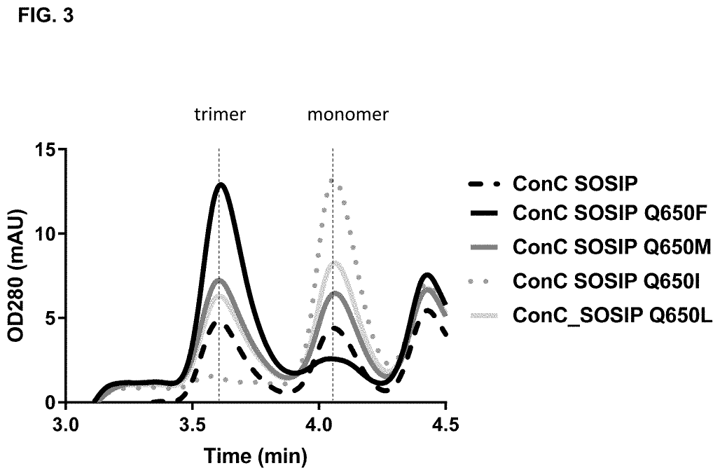

At position 650, a few other amino acid substitutions were tested besides tryptophan (W). Phenylalanine (F) increased trimer yield considerably, and also methionine (M) and leucine (L) increased trimer, whereas in surprising contrast isoleucine (I) decreases trimer formation, as shown using analytical SEC of Expi293F cell culture supernatants after transfection with plasmids coding for the respective HIV Env ConC_SOSIP variants (FIG. 3).

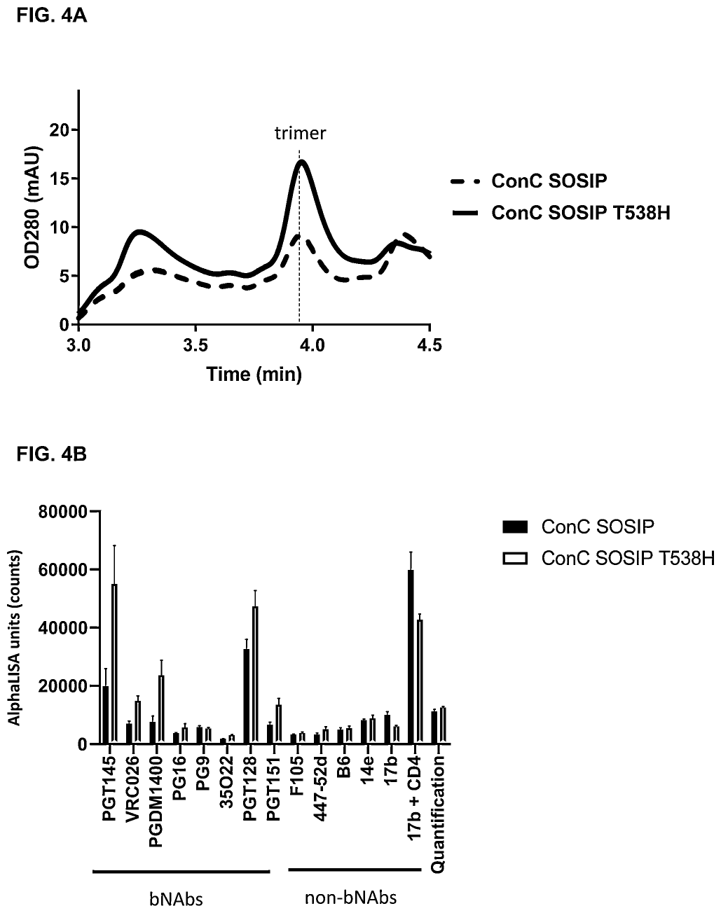

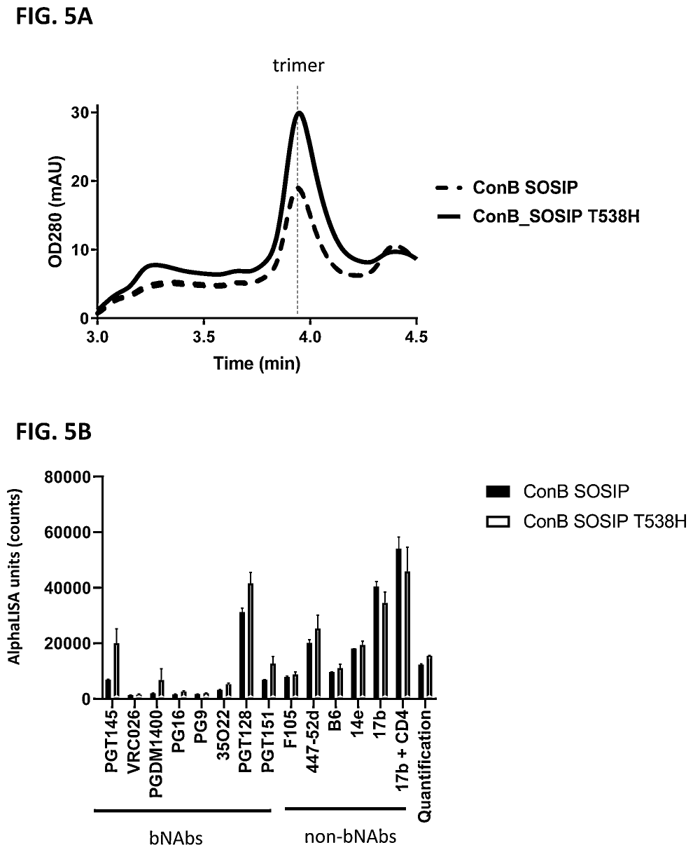

It was also shown that the mutation T538H increased trimer yield of both ConC_SOSIP and ConB-SOSIP (FIGS. 4A and 5A), and bNAb binding in AlphaLISA compared to its parent molecules not having this mutation. An increase in trimer-specific apex-directed broadly neutralizing antibodies (bNAbs) PGT145, VRCO26, and PGDM1400, was demonstrated for the T538H mutation, indicating improved trimer yield and/or trimer folding of ConC_SOSIP (FIG. 4B); the same observation was made for ConB_SOSIP, with the exception of VRCO26, which does not bind to this HIV Env irrespective of T538H stabilization (FIG. 5B). T538H reduces the binding of the non-bNAb 17b in AlphaLISA for both ConC_SOSIP and ConB_SOSIP (FIGS. 4B and 5B).

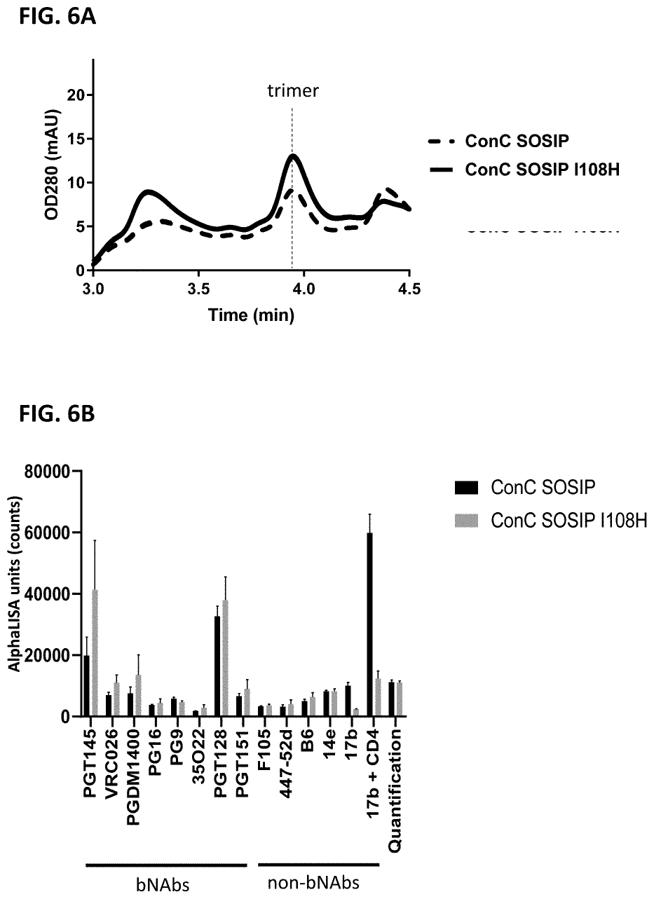

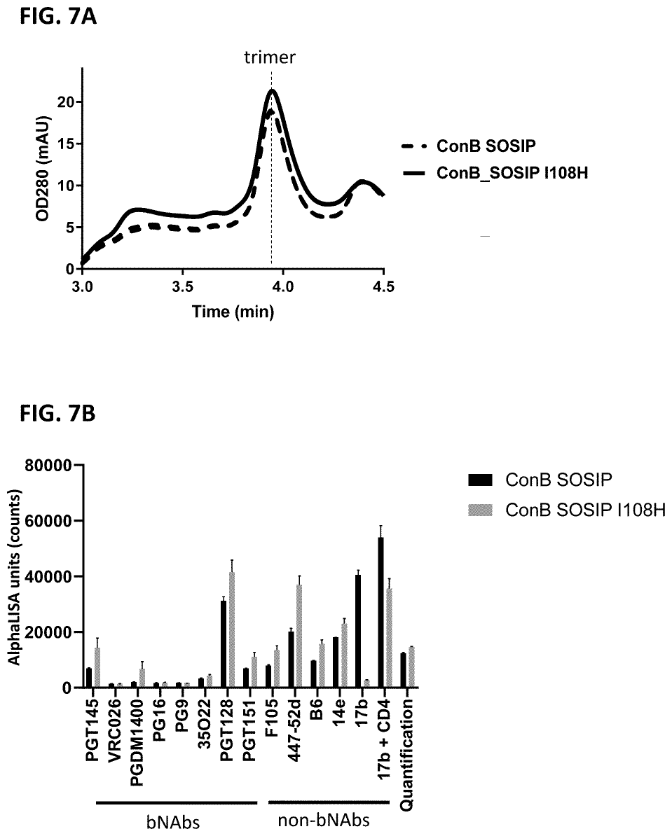

It was also shown that the mutation I108H increased trimer yield of both ConC_SOSIP and ConB-SOSIP (FIGS. 6A and 7A), and bNAb binding in AlphaLISA compared to its parent molecules not having this mutation. An increase in trimer-specific apex-directed broadly neutralizing antibodies (bNAbs) PGT145, VRCO26, and PGDM1400, was demonstrated for the I108H mutation, indicating improved trimer yield and/or trimer folding of ConC_SOSIP (FIG. 6B); the same observation was made for ConB_SOSIP, with the exception of VRCO26, which does not bind to this HIV Env irrespective of I108H stabilization (FIG. 7B). I108H strongly reduces the binding of the non-bNAb 17b in AlphaLISA for both ConC_SOSIP and ConB_SOSIP (FIGS. 6B and 7B).

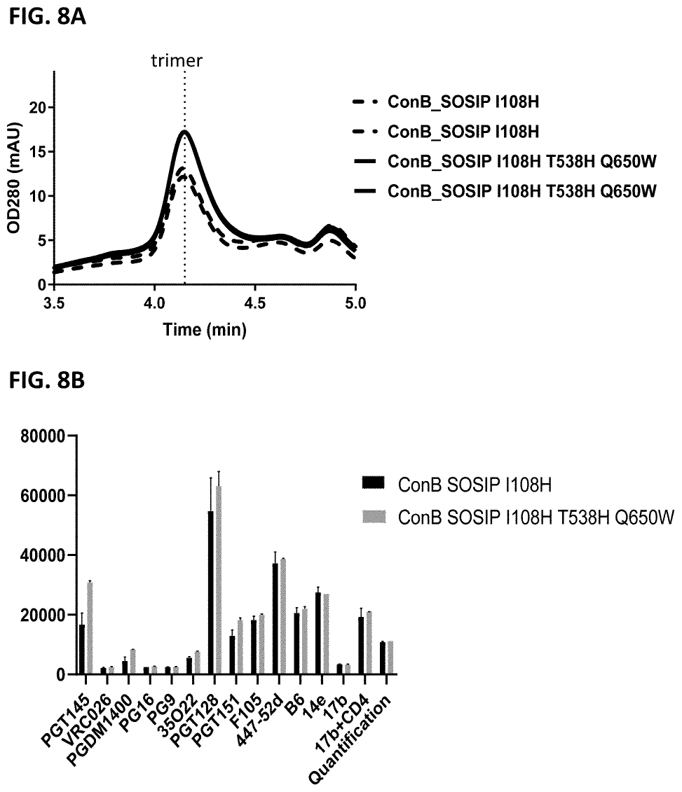

It was also shown that the combination of mutations I108H, T538H and Q650W increased trimer yield of ConB_SOSIP (FIG. 8A) and bNAb binding in AlphaLISA as compared to ConB_SOSIP comprising only I108H. An increase in trimer-specific apex-directed broadly neutralizing antibodies (bNAbs) PGT145 and PGDM1400, was demonstrated for ConB_SOSIP_I108H_T538H_Q650W indicating improved trimer yield and/or trimer folding as compared to ConB_SOSIP I108H (FIG. 8B). The same reduction in non-bNAb as measured by in AlphaLISA is observed for ConB_SOSIP I108H_T538H_Q650W as compared to ConB_SOSIP_I108H (FIG. 8B).

The mutation of position 650W, 650F, 650M, or 650L, preferably 650W or 650F, is also performed in HIV Env proteins from other clades, in natural HIV Env sequences, in HIV Env proteins not comprising one or all of the SOSIP mutations, in HIV Env proteins having one or more of the mutations indicated in entries (i)-(xvi) of Table 1, in HIV Env proteins having the T538H and/or the I108H mutation, and based upon the present application and the knowledge of the HIV Env protein it is plausible that each of the 650W, 650F, 650M, and 650L mutations, preferably 650W or 650F, also works in most or all of those backgrounds to increase trimer formation and/or trimer yield.

The data shown herein demonstrate that molecules of the invention, i.e. HIV Env proteins with a Trp, Phe, Met, or Leu, preferably Trp or Leu at position 650, have a surprisingly increased trimer formation and/or trimer yield as compared to HIV Env proteins with the naturally occuring amino acid at that position. The resulting Env trimers having Trp at position 650 have an increased propensity to be in a closed native prefusion conformation.

HIV envelope proteins having an increased percentage of trimer formation are advantageous from a manufacturing perspective, such as for vaccines, because less purification and removal of the envelope protein present in the preparation in the undesired non-native conformations will be required. Also, an increased total expression yield of the trimer is advantageous for manufacturing a vaccine product. HIV envelope proteins that are mainly in a closed native prefusion conformation are desirable for vaccination also because it is believed that they are structurally closer to Env proteins during actual infections, so that immune responses raised to Env proteins in such a conformation are highly beneficial.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

LIST OF SEQUENCES
gp160 of HIV-1 isolate HXB2 (signal sequence in italics; Ile at position 108,
Thr at position 538, and Gln at position 650 underlined and bold)
SEQ ID NO: 1
MRVKEKYQHLWRWGWRWGTMLLGMLMICSATEKLWVTVYYGVPVWKEATTTLFCASDAKAYDTEVHNVWATHACVPT
DPNPQEVVLVNVTENFNMWKNDMVEQMHED [symbol] ISLWDQSLKPCVKLTPLCVSLKCTDLKNDTNTNSSSGRMIMEKGEI
KNCSFNISTSIRGKVQKEYAFFYKLDIIPIDNDTTSYKLTSCNTSVITQACPKVSFEPIPIHYCAPAGFAILKCNNK
TFNGTGPCTNVSTVQCTHGIRPVVSTQLLLNGSLAEEEVVIRSVNFTDNAKTIIVQLNTSVEINCTRPNNNTRKRIR
IQRGPGRAFVTIGKIGNMRQAHCNISRAKWNNTLKQIASKLREQFGNNKTIIFKQSSGGDPEIVTHSFNCGGEFFYC
NSTQLFNSTWFNSTWSTEGSNNTEGSDTITLPCRIKQIINMWQKVGKAMYAPPISGQIRCSSNITGLLLTRDGGNSN
NESEIFRPGGGDMRDNWRSELYKYKVVKIEPLGVAPTKAKRRVVQREKRAVGIGALFLGFLGAAGSTMGAASMTL [symbol] V
QARQLLSGIVQQQNNLLRAIEAQQHLLQLTVWGIKQLQARILAVERYLKDQQLLGIWGCSGKLICTTAVPWNASWSN
KSLEQIWNHTTWMEWDREINNYTSLIHSLIEES [symbol] NQQEKNEQELLELDKWASLWNWFNITNWLWYIKLFIMIVGGLV
GLRIVFAVLSIVNRVRQGYSPLSFQTHLPTPRGPDRPEGIEEEGGERDRDRSIRLVNGSLALIWDDLRSLCLFSYHR
LRDLLLIVTRIVELLGRRGWEALKYWWNLLQYWSQELKNSAVSLLNATAIAVAEGTDRVIEVVQGACRAIRHIPRRI
RQGLERILL
HIV Env exemplary consensus clade C (consensus sequence only, not including
any signal sequence, transmembrane domain (664 is last amino acid), SOSIP
mutations, and/or furin cleavage site mutations; Ile at position 108, Thr
at position 538, and Gln at position 650 underlined and bold)
SEQ ID NO: 2
NLWVTVYYGVPVWKEAKTTLFCASDAKAYEKEVHNVWATHACVPTDPNPQEMVLENVTENFNMWKNDMVDQMHED [symbol] I
SLWDQSLKPCVKLTPLCVTLNCTNVNVTNTNNNNMKEEMKNCSENTTTEIRDKKQKEYALFYRLDIVPLNENSSEYR
LINCNTSTITQACPKVSFDPIPIHYCAPAGYAILKCNNKTFNGTGPCNNVSTVQCTHGIKPVVSTQLLLNGSLAEEE
IIIRSENLTDNAKTIIVHLNESVEINCTRPNNNTRKSIRIGPGQTFYATGDIIGDIRQAHCNISEAKWNKTLQRVKK
KLKEHFPNKTIKFAPSSGGDLEITTHSFNCRGEFFYCNTSKLFNSTYNNTTSNSTITLPCRIKQIINMWQEVGRAMY
APPIAGNITCKSNITGLLLTRDGGNNNNNTETFRPGGGDMRDNWRSELYKYKVVEIKPLGIAPTKAKRRVVEREKRR
AVGIGAVFLGELGAAGSTMGAASITL [symbol] VQARQLLSGIVQQQSNLLRAIEAQQHMLQLTVWGIKQLQARVLAIERYLK
DQQLLGIWGCSGKLICTTAVPWNSSWSNKSQEDIWDNMTWMQWDREISNYTDTIYRLLEES [symbol] NQQEKNEKDLLALD
ConC_SOSIP (mature clade C consensus sequence with SOSIP mutations and
furin cleavage site, and C-terminal truncation, and a sortase A-Flag-His tag
at the C-term (underlined); Ile at position 108, Thr at position 538, and
Gln at position 650 underlined and bold) (HIV150606)
SEQ ID NO: 3
NLWVTVYYGVPVWKEAKTTLFCASDAKAYEKEVHNVWATHACVPTDPNPQEMVLENVTENFNMWKNDMVDQMHED [symbol] I
SLWDQSLKPCVKLTPLCVTLNCTNVNVTNTNNNNMKEEMKNCSFNTTTEIRDKKQKEYALFYRLDIVPLNENSSEYR
LINCNTSTITQACPKVSFDPIPIHYCAPAGYAILKCNNKTFNGTGPCNNVSTVQCTHGIKPVVSTQLLLNGSLAEEE
IIIRSENLTDNAKTIIVHLNESVEINCTRPNNNTRKSIRIGPGQTFYATGDIIGDIRQAHCNISEAKWNKTLQRVKK
KLKEHFPNKTIKFAPSSGGDLEITTHSFNCRGEFFYCNTSKLFNSTYNNTTSNSTITLPCRIKQIINMWQEVGRAMY
APPIAGNITCKSNITGLLLTRDGGNNNNNTETFRPGGGDMRDNWRSELYKYKVVEIKPLGIAPTKCKRRVVERRRRR
RAVGIGAVFLGELGAAGSTMGAASITL [symbol] VQARQLLSGIVQQQSNLLRAPEAQQHMLQLTVWGIKQLQARVLAIERYL
KDQQLLGIWGCSGKLICCTAVPWNSSWSNKSQEDIWDNMTWMQWDREISNYTDTIYRLLEES [symbol] NQQEKNEKDLLALD
AAALPETGGGSDYKDDDDKPGGGGSGGGGSGGGGSGGGGSGGGGSGGGGSGGGGSHHHHHH
HIV Env exemplary consensus clade B (consensus sequence only, not including
any signal sequence, transmembrane domain (664 is last amino acid), SOSIP
mutations, and/or furin cleavage site mutations; Ile at position 108, Thr at
position 538, and Gln at position 650 underlined and bold)
SEQ ID NO: 4
AEKLWVTVYYGVPVWKEATTTLFCASDAKAYDTEVHNVWATHACVPTDPNPQEVVLENVTENFNMWKNNMVEQMHED [symbol]
ISLWDQSLKPCVKLTPLCVTLNCTDLNNNTTNNNSSSEKMEKGEIKNCSFNITTSIRDKVQKEYALFYKLDVVPID
NNNTSYRLISCNTSVITQACPKVSFEPIPIHYCAPAGFAILKCNDKKFNGTGPCTNVSTVQCTHGIRPVVSTQLLLN
GSLAEEEVVIRSENFTDNAKTIIVQLNESVEINCTRPNNNTRKSIHIGPGRAFYATGDIIGDIRQAHCNISRTKWNN
TLKQIVKKLREQFGNKTIVFNQSSGGDPEIVMHSFNCGGEFFYCNTTQLFNSTWNSNGTWNNTTGNDTITLPCRIKQ
IINMWQEVGKAMYAPPIRGQIRCSSNITGLLLTRDGGNNNNNTTETFRPGGGDMRDNWRSELYKYKVVKIEPLGVAP
TKCKRRVVQRRRRRRAVGIGAMFLGFLGAAGSTMGAASITL [symbol] VQARQLLSGIVQQQNNLLRAPEAQQHLLQLTVWGI
KQLQARVLAVERYLKDQQLLGIWGCSGKLICCTAVPWNTSWSNKSLDEIWDNMTWMQWEREIDNYTGLIYTLIEES [symbol]
NQQEKNEQELLELD
ConB_SOSIP (mature clade B consensus sequence with SOSIP mutations and
furin cleavage site, and C-terminal truncation, and a sortase A-Flag-His tag
at the C-term (underlined) ; Ile at position 108, Thr at position 538, and
Gln at position 650 underlined and bold) (HIV150599)
SEQ ID NO: 5
AEKLWVTVYYGVPVWKEATTTLFCASDAKAYDTEVHNVWATHACVPTDPNPQEVVLENVTENFNMWKNNMVEQMHED [symbol]
ISLWDQSLKPCVKLTPLCVTLNCTDLNNNTTNNNSSSEKMEKGEIKNCSFNITTSIRDKVQKEYALFYKLDVVPID
NNNTSYRLISCNTSVITQACPKVSFEPIPIHYCAPAGFAILKCNDKKFNGTGPCTNVSTVQCTHGIRPVVSTQLLLN
GSLAEEEVVIRSENFTDNAKTIIVQLNESVEINCTRPNNNTRKSIHIGPGRAFYATGDIIGDIRQAHCNISRTKWNN
TLKQIVKKLREQFGNKTIVFNQSSGGDPEIVMHSFNCGGEFFYCNTTQLFNSTWNSNGTWNNTTGNDTITLPCRIKQ
IINMWQEVGKAMYAPPIRGQIRCSSNITGLLLTRDGGNNNNNTTETFRPGGGDMRDNWRSELYKYKVVKIEPLGVAP
TKCKRRVVQRRRRRRAVGIGAMFLGFLGAAGSTMGAASITL [symbol] VQARQLLSGIVQQQNNLLRAPEAQQHLLQLTVWGI
KQLQARVLAVERYLKDQQLLGIWGCSGKLICCTAVPWNTSWSNKSLDEIWDNMTWMQWEREIDNYTGLIYTLIEES [symbol]
NQQEKNEQELLELDAAALPETGGGSDYKDDDDKPGGGGSGGGGSGGGGSGGGGSGGGGSGGGGSGGGGSHHHHHH
(furin cleavage site mutant sequence)
SEQ ID NO: 6
RRRRRR
(example of a signal sequence (e.g. used for ConC_SOSIP))
SEQ ID NO: 7
MRVRGILRNWQQWWIWGILGFWMLMICNVVG
(note: the last VG could be the beginning of the mature
protein or the end of the signal sequence)
(example of a signal sequence (e.g. used for ConB_SOSIP)
SEQ ID NO: 8
MRVKGIRKNYQHLWRWGTMLLGMLMICSA
(example of 8 amino acid sequence that can replace HR1 loop)
SEQ ID NO: 9
NPDWLPDM
(example of 8 amino acid sequence that can replace HR1 loop)
SEQ ID NO: 10
GSGSGSGS
(example of 8 amino acid sequence that can replace HR1 loop)
SEQ ID NO: 11
DDVHPDWD
(example of 8 amino acid sequence that can replace HR1 loop)
SEQ ID NO: 12
RDTFALMM
(example of 8 amino acid sequence that can replace HR1 loop)
SEQ ID NO: 13
DEEKVMDF
(example of 8 amino acid sequence that can replace HR1 loop)
SEQ ID NO: 14
DEDPHWDP
(sortase A-Flag-His tag)
SEQ ID NO: 15
AAALPETGGGSDYKDDDDKPGGGGSGGGGSGGGGSGGGGSGGGGSGGGGSGGGGSHHHHHH
(exemplary full length ConC_SOSIP (including signal sequence, in italics);
Ile at position 108, Thr at position 538, and Gln at position 650 underlined
and bold)
SEQ ID NO: 16
MRVRGILRNWQQWWIWGILGFWMLMICNVVGNLWVTVYYGVPVWKEAKTTLFCASDAKAYEKEVHNVWATHACVPTD
PNPQEMVLENVTENFNMWKNDMVDQMHED [symbol] ISLWDQSLKPCVKLTPLCVTLNCINVNVINTNNNNMKEEMKNCSFNT
TTEIRDKKQKEYALFYRLDIVPLNENSSEYRLINCNTSTITQACPKVSFDPIPIHYCAPAGYAILKCNNKTFNGTGP
CNNVSTVQCTHGIKPVVSTQLLLNGSLAEEEIIIRSENLTDNAKTIIVHLNESVEINCTRPNNNTRKSIRIGPGQTF
YATGDIIGDIRQAHCNISEAKWNKTLQRVKKKLKEHFPNKTIKFAPSSGGDLEITTHSFNCRGEFFYCNTSKLFNST
YNNTTSNSTITLPCRIKQIINMWQEVGRAMYAPPIAGNITCKSNITGLLLTRDGGNNNNNTETFRPGGGDMRDNWRS
ELYKYKVVEIKPLGIAPTKCKRRVVERekRAVGIGAVFLGFLGAAGSTMGAASITL [symbol] VQARQLLSGIVQQQSNLLRA
PEAQQHMLQLTVWGIKQLQARVLAIERYLKDQQLLGIWGCSGKLICCTAVPWNSSWSNKSQEDIWDNMTWMQWDREI
SNYTDTIYRLLEES [symbol] NQQEKNEKDLLALDSWNNLWNWFDITNWLWYIKIFIMIVGGLIGLRIIFAVLSIVNRVRQGY
SPLSFQTLTPNPRGPDRLGRIEEEGGEQDRDRSIRLVSGFLALAWDDLRSLCLFSYHRLRDFILIAARAVELLGRSS
LRGLQRGWEALKYLGSLVQYWGLELKKSAISLLDTIAIAVAEGTDRIIELIQRICRAIRNIPRRIRQGFEAALL

### REFERENCES

1. Sanders et al. J. Virol. (2002) 76(17), 8875-89
2. Sanders et al. Science (2015) 349(6224), 139-140
3. Julien et al. Proc. Nat. Acad. Sci. (2015) 112(38), 11947-52
4. de Taeye et al. Cell (2015) 163(7), 1702-15
5. Kwon et al. (2015) Nat. Struct. Mol. Biol. 22(7) 522-31
6. Eglen et al. Curr. Chem. Genomics, (2008) 25(1), 2-10
7. Kong et al, Nat Commun. 2016 June 28; 7:12040. doi: 10.1038/ncomms12040
8. Barouch et al, Nat Med 2010, 16: 319-323
9. WO 2010/059732
10. Sharma S K, et al. Cell Rep. (2015) 11(4):539-50. doi: 10.1016/j.celrep.2015.03.047.
11. Georgiev I S, et al. J Virol. (2015) 89(10):5318-29. doi: 10.1128/JVI.03451-14.
12. López-Sagaseta J, et al (2016) Computational and Struct Biotechnol J 14: 58-68.
13. Zhao L, et al (2014) Vaccine 32: 327-337
14. He L, et al (2016) Nat Commun. 2016 Jun. 28; 7:12041. doi: 10.1038/ncomms12041
15. WO2011082087
16. Bale S, et al (2017) J. Virol. doi:10.1128/JVI.00443-17
17. Abbink et al (2007) Virol. 81(9): 4654-64
18. Altschul S F et al (1997) Nucleic Acid Res. 25: 3389-3402
19. Harris et al (2011) PNAS 108 (28): 11440-11445
20. Kushnir et al (2012) Vaccine (31): 58-83
21. WO 2007/104792
22. WO 2014/124301
23. US 2016/0122392
24. WO 2018/050747
25. WO 2019/016062
26. WO 2017/102929
27. WO 2012/172277
28. WO 2018/215766
29. WO 2019/086466
30. WO 2019/086456
31. WO 96/09378
32. US2011/0300205
33. WO 2019/023566
34. Pardi et al, 2018, Nature Reviews Drug Discovery 17: 261-279
35. Zhang et al, 2019, Front. Immunol. 10: 594
36. Eglen et al. Curr. Chem. Genomics, (2008) 25(1), 2-10
37. Rutten et al, (2018) Cell Reports 23: 584-595
38. Lee et al, 2017, Immunity 46: 690-702
39. Rawi et al, (2020) Cell Reports 33, 108432

1. A recombinant human immunodeficiency virus (HIV) envelope (Env) protein comprising one of the amino acids tryptophan (Trp), phenylalanine (Phe), methionine (Met), or leucine (Leu) at position 650,
wherein the numbering of the positions is according to the numbering in gp160 of HIV-1 isolate HXB2.

2. The recombinant HIV Env protein of claim 1, further comprising one or more of the following amino acid residues at the indicated positions:
(i) Phe, Leu, Met, or Trp at position 651;
(ii) Phe, Ile, Met, or Trp at position 655;
(iii) Asn or Gln at position 535;
(iv) Val, Ile or Ala at position 589;
(v) Phe or Trp at position 573;
(vi) Ile at position 204;
(vii) Phe, Met, or Ile at position 647;
(viii) Val, Ile, Phe, Met, Ala, or Leu at position 658;
(ix) Gln, Glu, Ile, Met, Val, Trp, or Phe at position 588;
(x) Lys at position 64 or Arg at position 66 or Lys at position 64 and Arg at position 66;
(xi) Trp at position 316;
(xii) Cys at both positions 201 and 433;
(xiii) Pro at position 556 or 558 or at both positions 556 and 558;
(xiv) replacement of the loop at amino acid positions 548-568 (HR1-loop) by a loop having 7-10 amino acids;
(xv) Gly at position 568, or Gly at position 569, or Gly at position 636, or Gly at both positions 568 and 636, or Gly at both positions 569 and 636;
(xvi) Tyr at position 302, or Arg at position 519, or Arg at position 520, or Tyr at position 302 and Arg at position 519, or Tyr at position 302 and Arg at position 520, or Tyr at position 302 and Arg at both positions 519 and 520;
(xvii) a mutation in a furin cleavage sequence of the HIV Env protein;
(xviii) Cys at positions 501 and 605 or Pro at position 559, preferably Cys at positions 501 and 605 and Pro at position 559;
(xix) His at position 108; and/or
(xx) His at position 538,
wherein the numbering of the positions is according to the numbering in gp160 of HIV-1 isolate HXB2.

3. The recombinant HIV Env protein of claim 1, comprising Trp at position 650.

4. The recombinant HIV Env protein of claim 1, comprising Phe at position 650.

5. The recombinant HIV Env protein of claim 1, comprising His at position 108.

6. The recombinant HIV Env protein of claim 1, comprising His at position 538.

7. The recombinant HIV Env protein of claim 1, comprising Cys at positions 501 and 605 or Pro at position 559.

8. The recombinant HIV Env protein of claim 1, comprising Cys at positions 501 and 605 and Pro at position 559.

9. The recombinant HIV Env protein of claim 1, being a gp140 or gp160 protein, or an Env protein having a truncation in the cytoplasmic region.

10. The recombinant HIV Env protein of claim 1, which is an Env protein of a clade A HIV, a clade B HIV, or a clade C HIV.

11. A trimeric complex comprising a noncovalent oligomer of three identical recombinant HIV Env proteins of claim 1.

12. A particle displaying on its surface a recombinant HIV Env protein of claim 1.

13. An isolated nucleic acid molecule encoding a recombinant HIV Env protein of claim 1.

14. A vector comprising the isolated nucleic acid molecule of claim 13 operably linked to a promoter.

15. The vector of claim 14, which is an adenovirus vector.

16. A host cell comprising the isolated nucleic acid molecule of claim 13.

17. A method of producing a recombinant HIV Env protein, comprising growing the host cell of claim 16 under conditions suitable for production of the recombinant HIV Env protein.

18. A composition comprising the recombinant HIV Env protein of claim 1 and a pharmaceutically acceptable carrier.

19. A method of improving the trimer formation of an HIV Env protein, the method comprising substituting an amino acid residue at position 650 in a parent HIV Env protein by one of Trp, Phe, Met, or Leu, wherein the numbering of the positions is according to the numbering in gp160 of HIV-1 isolate HXB2.

20. A recombinant human immunodeficiency virus (HIV) envelope (Env) protein comprising histidine (His) at position 108, wherein the numbering of the positions is according to the numbering in gp160 of HIV-1 isolate HXB2.
